(19) United States
(12) Patent Application Publication
(10) Pub. No.: US 2025/0296011 A1
(43) Pub. Date: Sep. 25, 2025

(54) ORGANIC SOLVENT COLLECTION APPARATUS, SUBSTRATE PROCESSING APPARATUS, AND ORGANIC SOLVENT COLLECTION METHOD

(71) Applicant: SCREEN Holdings Co., Ltd., Kyoto (JP)

(72) Inventors: Michinori IWAO, Kyoto-shi (JP); Yukifumi YOSHIDA, Kyoto-shi (JP); Tomohiro UEMURA, Kyoto-shi (JP); Shoyo MINAMI, Kyoto-shi (JP); Yusuke UEDA, Kyoto-shi (JP)

(21) Appl. No.: 19/070,512

(22) Filed: Mar. 4, 2025

(30) Foreign Application Priority Data
Mar. 21, 2024 (JP) 2024-044775

(51) Int. Cl. B01D 3/14; B01D 17/00; B01D 17/12; C02F 1/04; C02F 1/44; C02F 101/34; C02F 103/34; H01L 21/67

(52) U.S. Cl. CPC B01D 3/145; B01D 17/085; B01D 17/12; C02F 1/04; C02F 1/44; C02F 2101/34; C02F 2103/346; C02F 2201/005; C02F 2301/046; H01L 21/67017

(57) ABSTRACT

An organic solvent collection apparatus includes a collection pipe, a first dewaterer, and a second dewaterer. A mixed liquid of an organic solvent and water discharged from a processing unit flows through the collection pipe. The first dewaterer includes a first membrane separator that includes a first separation membrane having an application range of a solvent concentration and separates water from the mixed liquid having a solvent concentration greater than or equal to a concentration lower limit value of the application range. The second dewaterer is provided at a preceding stage of the first dewaterer, separates water from the mixed liquid discharged through the collection pipe to increase the solvent concentration of the mixed liquid to greater than or equal to the concentration lower limit value, and supplies the mixed liquid having the solvent concentration greater than or equal to the concentration lower limit value to the first dewaterer.

### BACKGROUND

### Technical Field

The present disclosure relates to an organic solvent collection apparatus, a substrate processing apparatus, and an organic solvent collection method.

### Description of the Background Art

Japanese Patent Application Laid-Open No. 2017-41505 discloses an IPA collection system that collects water-containing IPA (isopropyl alcohol) discharged from a processing unit processing a substrate. The IPA collection system includes a storage tank, a circulation pipe, a pump, a dewatering unit, and a filter. Water-containing IPA from the processing unit is supplied to the storage tank. The circulation pipe is connected to the storage tank, and returns the water-containing IPA from the storage tank to the storage tank. The pump is provided in the circulation pipe, and sends the water-containing IPA from an upstream end toward a downstream end of the circulation pipe. The filter is provided in the circulation pipe to remove foreign substances in the water-containing IPA. The dewatering unit is provided in the circulation pipe to remove moisture from the water-containing IPA.

The IPA collection system circulates the water-containing IPA through a circulation path including the storage tank and the circulation pipe. This circulation causes the water-containing IPA to pass through the filter and the dewatering unit. Thus, an IPA concentration of the water-containing IPA during the circulation increases, and the foreign matters in the water-containing IPA are reduced. That is, by this circulation, the clean water-containing IPA having the high IPA concentration is stored in the storage tank. The water-containing IPA in this storage tank is again supplied to the processing unit. Thus, an amount of discarded IPA can be reduced.

As the dewatering unit that separates water from a mixed liquid, it is conceivable to use a membrane separator including a separation membrane. The separation membrane is a membrane that allows the water to pass therethrough and substantially blocks an organic solvent. Such a membrane separator can separate the water from the mixed liquid with high energy efficiency as compared with, for example, a separation method such as distillation. On the other hand, in the case where the low concentration of the organic solvent in the mixed liquid is low, there is a risk that a problem is generated in the membrane separator when the mixed liquid is caused to flow into the membrane separator.

### SUMMARY

One aspect is an organic solvent collection apparatus including: a collection pipe through which a mixed liquid of an organic solvent and water discharged from a processing unit that processes a substrate flows; a first dewaterer including a first membrane separator including a first separation membrane having an application range of a solvent concentration, the first membrane separator separating water from the mixed liquid having the solvent concentration greater than or equal to a concentration lower limit value that is a lower limit value of the application range to increase the solvent concentration of the mixed liquid; and a second dewaterer that is provided at a preceding stage of the first dewaterer, the second dewaterer separating water from the mixed liquid discharged through the collection pipe to increase the solvent concentration of the mixed liquid to greater than or equal to the concentration lower limit value, and supplying the mixed liquid having the solvent concentration greater than or equal to the concentration lower limit value to the first dewaterer.

Another aspect is a substrate processing apparatus including the organic solvent collection apparatus and the processing unit.

Another aspect is an organic solvent collection method including: a first step of separating water from a mixed liquid of an organic solvent and water discharged from a processing unit that processes a substrate to increase a solvent concentration of the mixed liquid; and a second step of separating water from the mixed liquid using a first membrane separator including a first separation membrane having an application range of the solvent concentration after the first step to increase the solvent concentration of the mixed liquid, in which, in the first step, the solvent concentration of the mixed liquid is increased greater than or equal to a concentration lower limit value that is a lower limit value of the application range of the first separation membrane.

These and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is a plan view schematically illustrating an example of a substrate processing apparatus;

FIG. 2 is a side view schematically illustrating an example of a processing unit;

FIG. 3 is a view schematically illustrating an example of an organic solvent collection part according to a first embodiment;

FIG. 4 is a view schematically illustrating an example of a specific configuration of the organic solvent collection part;

FIG. 5 is a flowchart illustrating an example of an operation of the organic solvent collection part;

FIG. 6 is a view schematically illustrating an example of a second dewaterer according to a second embodiment;

FIG. 7 is a view schematically illustrating an example of an organic solvent collection part according to a third embodiment;

FIG. 8 is a view schematically illustrating a first example of a more specific configuration of the organic solvent collection part according to the third embodiment;

FIG. 9 is a view schematically illustrating a second example of the more specific configuration of the organic solvent collection part according to the third embodiment;

FIG. 10 is a view schematically illustrating a first example of a substrate processing apparatus according to a fourth embodiment;

FIG. 11 is a flowchart illustrating an example of an operation of an organic solvent collection part according to the fourth embodiment;

FIG. 12 is a view schematically illustrating an example of a processing unit according to the fourth embodiment;

FIGS. 13A to 13F are views schematically illustrating examples of states of the processing unit in respective steps of Table 1;

FIG. 14 is a graph illustrating an example of a distance from a center of a substrate at each position on the substrate and a thickness of a liquid film of pure water at each position;

FIGS. 15A to 15E are views schematically illustrating examples of the states of the processing unit in respective steps of Table 2;

FIG. 16 is a graph illustrating an example of the distance from the center of the substrate at each position on the substrate and the thickness of the liquid film at each position;

FIG. 17 is a flowchart illustrating an example of an operation of a concentration estimator;

FIG. 18 is a view schematically illustrating a second example of the substrate processing apparatus according to the fourth embodiment; and

FIG. 19 is a view schematically illustrating an example of a substrate processing apparatus according to a fifth embodiment.

### DESCRIPTION OF THE PREFERRED EMBODIMENTS

Hereinafter, embodiments will be described in detail with reference to the drawings. In the drawings, dimensions and numbers of each part are exaggerated or simplified as necessary for easy understanding. Portions having similar configurations and functions are denoted by the same reference numeral, and redundant description will be omitted in the following description.

In the following description, the same components are denoted by the same reference numeral, and it is assumed that names and functions of the same components are also similar. Consequently, the detailed description of the same component is occasionally omitted in order to avoid duplication.

In the following description, even when ordinal numbers such as “first” or “second” are used, these terms are used only for convenience to facilitate understanding of contents of the embodiments, and are not limited to the order that can be generated by these ordinal numbers.

In the case where expressions indicating a relative or absolute positional relationship (for example, “in one direction”, “along one direction”, “parallel”, “orthogonal”, “center”, “concentric”, and “coaxial”) are used, the expressions shall not only strictly represent a positional relationship, but also represent a state of being displaced relative to an angle or a distance to an extent that a tolerance or a comparable function is obtained, unless otherwise specified. When expressions indicating an equal state (for example, “same”, “equal”, and “homogeneous”) are used, unless otherwise specified, the expressions shall not only represent a quantitatively strictly equal state, but also represent a state in which there is a difference in obtaining a tolerance or a similar function. In the case where expressions indicating a shape (for example, “quadrangular” or “cylindrical”) are used, unless otherwise specified, the expressions shall not only represent the shape geometrically and strictly, but also represent a shape having, for example, unevenness or chamfering within a range in which the same level of effect can be obtained. When expressions “comprising”, “owning”, “possessing”, “including” or “having” one component are used, the expressions are not an exclusive expression excluding presence of other components. When the expression “at least any one of A, B, and C” is used, the expression includes only A, only B, only C, any two of A, B and C, and all of A, B and C.

### First Embodiment

### <1. Substrate Processing Apparatus>

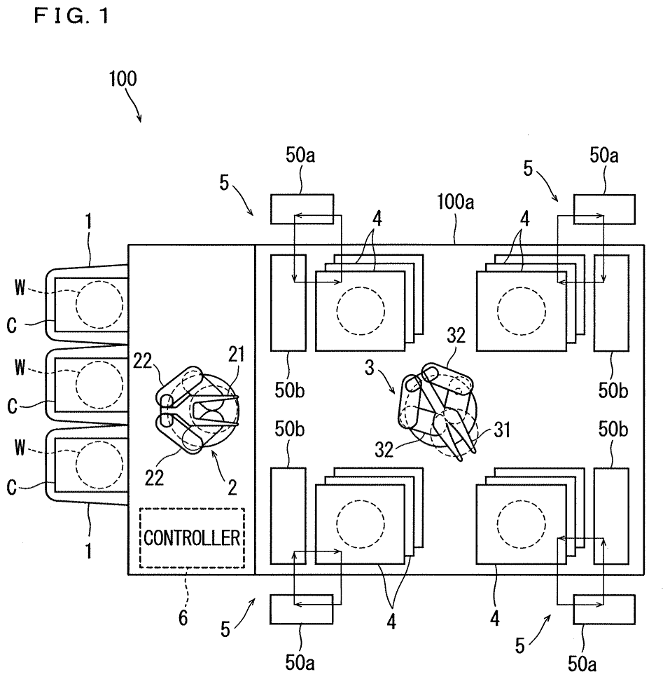

A substrate processing apparatus 100 according to an embodiment will be described with reference to FIG. 1. FIG. 1 is a plan view schematically illustrating an example of the substrate processing apparatus 100.

The substrate processing apparatus 100 is a single wafer type processing apparatus that processes a substrates W, which is a processing target, one by one. For example, the substrate W that is the processing target processed by the substrate processing apparatus 100 is a semiconductor substrate. For example, the shape of the substrate W that is the processing target is a disk shape.

The substrate processing apparatus 100 includes a load port 1, an indexer robot 2, a main conveyance robot 3, a processing unit 4, an organic solvent collection part 5, and a controller 6.

The load port 1 is an interface taking in and out the substrate W with respect to a carrier C that is a kind of storage container storing a plurality of substrates. For example, a plurality of (three in the example of the drawing) load ports 1 are provided. For example, the plurality of load ports 1 are arrayed in a line in a horizontal direction. The carrier C may be of a type in which the substrate W is stored in a sealed space (for example, a front opening unified pod (FOUP), a standard mechanical interface (SMIF) pod, or the like), or may be of a type in which the substrate W is exposed to outside air (for example, an open cassette (OC)).

The indexer robot 2 is a conveyance apparatus that conveys the substrate W. As an example, the indexer robot 2 is a horizontal articulated robot, and includes a pair of hands 21, 21 holding the substrate W and an arm 22 connected to each hand 21. In addition, the indexer robot 2 includes a drive mechanism (not illustrated) that turns each hand 21 and bends and stretches, turns, and lifts and lowers each arm 22. The indexer robot 2 conveys the substrate W between the carrier C held in the load port 1 and the main conveyance robot 3. That is, the indexer robot 2 accesses the carrier C placed in the load port 1 and performs a carry-out operation (that is, the operation of taking out the substrate W stored in the carrier C with the hand 21) and a carry-in operation (that is, the operation of accommodating the substrate W held by the hand 21 in the carrier C). Furthermore, the indexer robot 2 accesses a transfer position and transfers the substrate W to and from the main conveyance robot 3.

The main conveyance robot 3 is a conveyance apparatus that conveys the substrate W. As an example, the main conveyance robot 3 is a horizontal articulated robot, and includes a pair of hands 31, 31 holding the substrate W and an arm 32 connected to each hand 31. In addition, the main conveyance robot 3 includes a drive mechanism (not illustrated) that turns each hand 31 and bends and stretches, turns, and lifts and lowers each arm 32. The main conveyance robot 3 conveys the substrate W between the indexer robot 2 and each processing unit 4. That is, the main conveyance robot 3 accesses the transfer position and transfers the substrate W to and from the indexer robot 2. The main conveyance robot 3 accesses the processing unit 4 and performs the carry-in operation (that is, an operation of carrying the substrate W held by the hand 31 in the processing unit 4) and the carry-out operation (that is, the operation of carrying out the substrate W in the processing unit 4 with the hand 31).

The processing unit 4 performs predetermined processing on the substrate W using a processing liquid (for example, a chemical liquid, a rinse liquid, and IPA). At this point, for example, a plurality of (for example, three) processing units 4 stacked in a vertical direction configure one tower, and the plurality of (for example, four in the example of the drawing) towers are provided so as to surround the main conveyance robot 3. A specific configuration of the processing unit 4 will be described later.

The organic solvent collection part 5 collects an organic solvent from the processing unit 4, purifies the collected organic solvent, and supplies the organic solvent to the processing unit 4 again. As an example, the organic solvent collection part 5 may be provided in one-to-one correspondence with each of the plurality of towers, and each organic solvent collection part 5 may collect and supply the organic solvent to each processing unit 4 included in the corresponding tower. A specific configuration of the organic solvent collection part 5 will be described later.

The controller 6 controls the operation of each unit (the load port 1, the indexer robot 2, the main conveyance robot 3, the processing unit 4, and the organic solvent collection part 5) included in the substrate processing apparatus 100. For example, the controller 6 is configured by a general computer having an electric circuit. As an example, the controller 6 includes a central processor unit (CPU) as a central processing unit that performs various types of arithmetic processing (data processing), a read only memory (ROM) that stores a basic program and the like, a random access memory (RAM) that is used as a work area when the CPU performs predetermined processing (data processing), a storage device configured by a nonvolatile storage device such as a flash memory or a hard disk device, a bus line that connects these, and the like. A program that defines processing executed by the controller 6 may be stored in the storage device, the RAM, or the like. In this case, for example, when the CPU executes the program, each part of the substrate processing apparatus 100 may be controlled by the controller 6, and the processing defined by the program may be executed in the substrate processing apparatus 100. That is, when the CPU executes the program, a circuit that performs the processing defined by the program may be implemented by the controller 6. However, a part or all of the control performed by the controller 6 (a part or all of the circuit implemented by the controller 6) may be executed (implemented) by hardware such as a dedicated logic circuit.

### <2. Processing Unit>

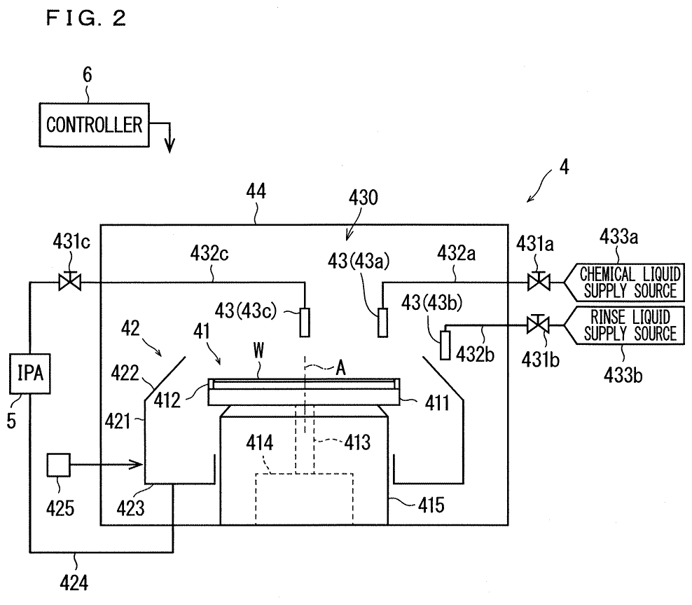

The processing unit 4 will be described with reference to FIG. 2. FIG. 2 is a side view schematically illustrating an example of the processing unit 4.

### <2-1. Configuration of Processing Unit>

The processing unit 4 performs predetermined processing on the substrate W using a processing liquid (for example, a chemical liquid, a rinse liquid, and IPA). For example, the processing unit 4 includes a spin chuck 41, which is an example of the substrate holder, a cup 42, and a dispenser 430. The dispenser 430 includes a nozzle 43. The spin chuck 41, the cup 42, and the nozzle 43 are accommodated in the processing chamber 44.

The spin chuck 41 rotates the substrate W about an axis (rotational axis) A extending vertically through a center of a main surface while holding the substrate W in a horizontal posture (a posture in which a thickness direction of the substrate W is along an up and down direction (vertical direction)). Specifically, for example, the spin chuck 41 includes a spin base 411. The spin base 411 is a disk-shaped member, and is disposed in a posture in which the thickness direction is along the up and down direction. A plurality of chuck pins 412 are provided on an upper surface of the spin base 411. The plurality of chuck pins 412 is disposed at equal intervals along a circumference corresponding to a peripheral edge of the substrate W. A link mechanism (not illustrated) that moves the chuck pins 412 between an abutting position and an open position is connected to the plurality of chuck pins 412. The “abutting position” is a position at which the chuck pin 412 abuts on the peripheral edge of the substrate W. The “open position” is a position where the chuck pin 412 is away from the peripheral edge of the substrate W. When each of the plurality of chuck pins 412 is disposed at the abutting position, the substrate W is held (chucked) above the spin base 411 in a horizontal posture. When each of the plurality of chuck pins 412 is disposed at the open position, the holding of the substrate W is released. The link mechanism selects the position of the chuck pin 412 according to an instruction from the controller 6. That is, timing of holding the substrate W, timing of releasing the holding of the substrate W, and the like are controlled by the controller 6. In addition, the spin base 411 is connected to a spin motor 414 through a shaft part 413 provided coaxially with the rotational axis A. The shaft part 413 and the spin motor 414 are accommodated in a cover 415. The spin motor 414 rotates the shaft part 413 around the rotational axis A. Thus, the spin base 411 and thus the substrate W held above the spin base 411 rotate around the rotational axis A. The spin motor 414 rotates the spin base 411 according to the instruction from the controller 6. That is, a rotation speed, rotation start timing, rotation end timing, and the like of the spin base 411 (and thus the substrate W) are controlled by the controller 6.

The cup 42 has a tubular shape surrounding the spin chuck 41, and catches the processing liquid discharged from the substrate W held and rotated by the spin chuck 41. Specifically, for example, the cup 42 includes a cylindrical guide part 421 disposed coaxially with the rotational axis A, an inclined part 422 that is continuous with an upper end of the guide part 421 and reduces in diameter upward, and a liquid receiver 423 that is continuous with a lower end of the guide part 421 and forms an annular groove opened upward. A cup-side collection pipe (specifically, for example, a cup-side collection pipe (not illustrated) for a chemical solution and a cup-side collection pipe 424 for IPA are used) that collects the liquid caught by the liquid receiver 423 are provided in the liquid receiver 423. In addition, a cup lifting mechanism 425 that lifts and lowers the cup 42 between a lower position and an upper position is connected to the cup 42. The “lower position” is a position where the upper end (specifically, the upper end of the inclined part 422) of the cup 42 is disposed below the substrate W held by the spin chuck 41. The “upper position” is a position where the upper end of the cup 42 is disposed above the substrate W held by the spin chuck 41. The cup lifting mechanism 425 moves up and down the cup 42 according to the instruction from the controller 6. That is, the position of the cup 42 is controlled by the controller 6.

The dispenser 430 (specifically, the nozzle 43) dispenses the processing liquid toward the upper surface of the substrate W held by the spin chuck 41. At this point, for example, the individual nozzle 43 is provided for each type of processing liquid. That is, the nozzle 43 that dispenses a chemical solution (hereinafter, also referred to as a “chemical liquid nozzle 43a”), the nozzle 43 that dispenses the rinse liquid (hereinafter, also referred to as a “rinse liquid nozzle 43b”), and the nozzle 43 that dispenses the IPA (hereinafter, also referred to as an “IPA nozzle 43c”) are provided.

The chemical liquid nozzle 43a dispenses the chemical liquid toward the upper surface of the substrate W held by the spin chuck 41. The chemical liquid nozzle 43a is connected to a chemical liquid supply source 433a through a chemical liquid pipe 432a in which a chemical liquid valve 431a is inserted. When the chemical liquid valve 431a is opened, the chemical liquid is supplied to the chemical liquid nozzle 43a through the chemical liquid pipe 432a, and the chemical liquid is dispensed from the chemical liquid nozzle 43a. The chemical liquid valve 431a is opened and closed according to the instruction from the controller 6. That is, the dispense timing of the chemical liquid from the chemical liquid nozzle 43a is controlled by the controller 6. For example, the chemical liquid is hydrofluoric acid. However, the chemical liquid is not limited to the hydrofluoric acid, but may be a liquid containing at least one of sulfuric acid, acetic acid, nitric acid, hydrochloric acid, hydrofluoric acid, phosphoric acid, ammonia water, hydrogen peroxide water, an organic acid (for example, citric acid or oxalic acid), an organic alkali (for example, tetramethylammonium hydroxide (TMAH)), a surfactant, and a corrosion inhibitor.

The rinse liquid nozzle 43b dispenses the rinse liquid toward the upper surface of the substrate W held by the spin chuck 41. The rinse liquid nozzle 43b is connected to the rinse liquid supply source 433b through the rinse liquid pipe 432b in which the rinse liquid valve 431b is inserted. When the rinse liquid valve 431b is opened, the rinse liquid is supplied to the rinse liquid nozzle 43b through the rinse liquid pipe 432b, and the rinse liquid is dispensed from the rinse liquid nozzle 43b. The rinse liquid valve 431b is opened and closed according to the instruction from the controller 6. That is, the dispense timing of the rinse liquid from the rinse liquid nozzle 43b is controlled by the controller 6. For example, the rinse liquid is pure water (deionized water). However, the rinse liquid is not limited to the pure water, but may be any of carbonated water, electrolyzed ion water, hydrogen water, ozone water, and hydrochloric acid water having a dilution concentration (for example, about 10 ppm to about 100 ppm).

The IPA nozzle 43c dispenses IPA (that is, a liquid containing the IPA as a main component) toward the upper surface of the substrate W held by the spin chuck 41. The IPA nozzle 43c is connected to the organic solvent collection part 5 through the IPA pipe 432c in which the IPA valve 431c is inserted. When the IPA valve 431c is opened, the IPA is supplied to the IPA nozzle 43c through the IPA pipe 432c, and the IPA is dispensed from the IPA nozzle 43c. The IPA valve 431c is opened and closed according to the instruction from the controller 6. That is, the dispense timing of the IPA from the IPA nozzle 43c is controlled by the controller 6.

A nozzle moving mechanism that moves the chemical liquid nozzle 43a, the rinse liquid nozzle 43b, and the IPA nozzle 43c between a processing position and a retracted position may be connected to at least one of the chemical liquid nozzle 43a, the rinse liquid nozzle 43b, and the IPA nozzle 43c. The “processing position” is a position where the processing liquid dispensed from the nozzles 43a, 43b, 43c is supplied to the substrate W held by the spin chuck 41. The “retracted position” is a position where the nozzles 43a, 43b, 43c are located outside (radially outside) the peripheral edge of the substrate W held by the spin chuck 41 when viewed from above. In this case, the nozzle moving mechanism moves the nozzles 43a, 43b, 43c according to the instruction from the controller 6. That is, the positions of the nozzles 43a, 43b, 43c are controlled by the controller 6.

### <2-2. Operation of Processing Unit>

An example of the operation of the processing unit 4 will be described. The operation performed by the processing unit 4 is performed under the control of the controller 6 (that is, the controller 6 controls the chuck pin 412, the spin motor 414, the cup lifting mechanism 425, the chemical liquid valve 431a, the rinse liquid valve 431b, the IPA valve 431c, and the like).

When the substrate W is carried in the processing chamber 44 by the main conveyance robot 3, the spin chuck 41 holds the substrate W. Subsequently, the spin chuck 41 starts the rotation.

In this state, the chemical liquid valve 431a is open. Then, the chemical liquid is dispensed from the chemical liquid nozzle 43a toward the upper surface of the substrate W held and rotated by the spin chuck 41. Thus, the chemical liquid is supplied to the entire upper surface of the substrate W, and the substrate W is processed by the chemical liquid (chemical liquid processing step). For example, when the hydrofluoric acid is used as the chemical solution, foreign substances such as particles are removed from the substrate W. The cup 42 is disposed at the upper position during the chemical liquid processing step. Consequently, the chemical liquid scattered around the substrate W is caught by the cup 42. That is, the chemical liquid scattered around the substrate W is caught by the inclined part 422, guided downward by the guide part 421, and collected in the liquid receiver 423. The chemical liquid (that is, the chemical liquid collected in the liquid receiver 423) caught by the cup 42 is collected through the cup-side collection pipe (not illustrated) for the chemical liquid.

The chemical liquid valve 431a is closed at a time point when a predetermined time elapses from the start of the dispense of the chemical solution. Then, the dispense of the chemical liquid from the chemical liquid nozzle 43a is stopped. Subsequently, the rinse liquid valve 431b is open. Then, the rinse liquid is dispensed from the rinse liquid nozzle 43b toward the upper surface of the substrate W held and rotated by the spin chuck 41. Thus, the rinse liquid is supplied to the entire upper surface of the substrate W, and the chemical liquid adhering to the substrate W is washed away by the rinse liquid (rinsing step). The cup 42 is also disposed at the upper position during the rinsing step. Consequently, the chemical liquid and the rinse liquid that are scattered around the substrate W are caught by the cup 42. The chemical liquid and the rinse liquid that are caught by the cup 42 are collected through the cup-side collection pipe (not illustrated) for the chemical liquid.

The rinse liquid valve 431b is closed at the time point when the predetermined time elapses from the start of the dispense of the rinse liquid. Then, the dispense of the rinse liquid from the rinse liquid nozzle 43b is stopped. Subsequently, the IPA valve 431c is open. Then, the IPA is dispensed from the IPA nozzle 43c toward the upper surface of the substrate W held and rotated by the spin chuck 41. Thus, the IPA is supplied to the entire upper surface of the substrate W, and the rinse liquid attached to the substrate W is replaced with the IPA (IPA supply step). The cup 42 is also disposed at the upper position during the IPA supply step. Consequently, the rinse liquid and the IPA that are scattered around the substrate W are caught by the cup 42. The rinse liquid and IPA that are caught by the cup 42 are collected through the cup-side collection pipe 424 for IPA.

The IPA valve 431c is closed at the time point when the predetermined time elapses from the start of the supply of IPA. Then, the dispense of the IPA from the IPA nozzle 43c is stopped. At this stage, the rinse liquid on the substrate W is completely replaced with the IPA, and a liquid film of the IPA covering the entire upper surface of the substrate W is formed. Subsequently, the spin chuck 41 starts high-speed rotation. Thus, the substrate W is rotated at a high speed, and the IPA on the substrate W is shaken off around the substrate W by centrifugal force (spin dry step). The cup 42 is also disposed at the upper position while the substrate W is rotated at the high speed. Consequently, the IPA scattered around the substrate W is caught by the cup 42. The IPA caught by the cup 42 is collected through the cup-side collection pipe 424 for IPA.

When a predetermined time elapses since the start of the high-speed rotation of the spin chuck 41, the rotation of the spin chuck 41 is stopped. At this stage, the IPA is removed from the substrate W, and the substrate W is dried. The dried substrate W is carried out of the processing chamber 44 by the main conveyance robot 3.

Thus, a series of pieces of processing on one substrate W is completed. In the processing unit 4, the series of operations described above is repeated, so that the plurality of substrates W are processed one by one.

### <3. Outline of Organic Solvent Collection Part 5 (Organic Solvent Collection Apparatus)>

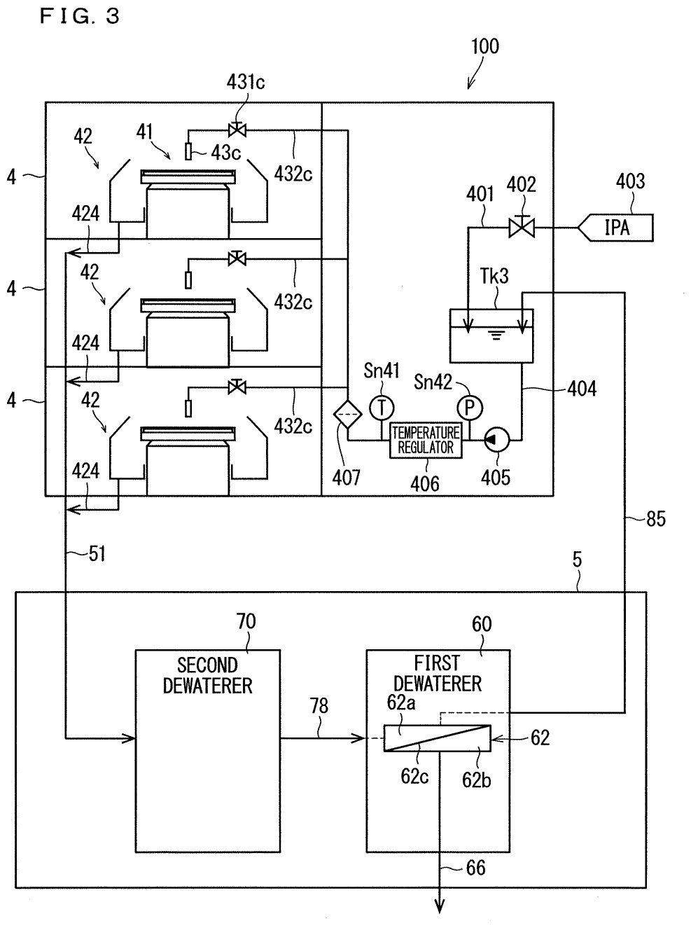

The configuration of the organic solvent collection part 5 will be described with reference to FIG. 3. FIG. 3 is a view schematically illustrating an example of the organic solvent collection part 5 according to the first embodiment. Hereinafter, an outline of the organic solvent collection part 5 will be first described, and then each configuration of the organic solvent collection part 5 will be described in detail.

The organic solvent collection part 5 includes a first dewaterer 60 and a second dewaterer 70. The second dewaterer 70 is provided at a preceding stage of the first dewaterer 60. A downstream end of a collection pipe 51 is connected to the second dewaterer 70. The collection pipe 51 is connected to the downstream end of each of the cup-side collection pipes 424. A mixed liquid of the organic solvent and water that are discharged from the processing unit 4 flows through the collection pipe 51. The organic solvent is, for example, an organic solvent having higher volatility or an organic solvent having lower surface tension than the water, and is, as a specific example, isopropyl alcohol (IPA). The mixed liquid is supplied (collected) to the second dewaterer 70 through the collection pipe 51. A buffer tank may be provided between the second dewaterer 70 and the processing unit 4. That is, the mixed liquid discharged from the processing unit 4 may be temporarily stored in the buffer tank, and the mixed liquid may be supplied from the buffer tank to the second dewaterer 70 through the collection pipe 51.

At this point, it is assumed that the concentration of the organic solvent (hereinafter, referred to as a solvent concentration) in the mixed liquid discharged from the processing unit 4 is low. As an example, the solvent concentration of the mixed liquid discharged from the processing unit 4 is less than or equal to 30 wt %.

The second dewaterer 70 separates the water from the mixed liquid to increase the solvent concentration of the mixed liquid. An example of a specific configuration of the second dewaterer 70 will be described in detail later. The second dewaterer 70 supplies the mixed liquid having the increased solvent concentration to the first dewaterer 60 through a first liquid sending pipe 78. An upstream end of the first liquid sending pipe 78 is connected to the second dewaterer 70, and the downstream end of the first liquid sending pipe 78 is connected to the first dewaterer 60.

The first dewaterer 60 includes a first membrane separator 62. The mixed liquid from the second dewaterer 70 flows into the first membrane separator 62. The first membrane separator 62 separates the water from the mixed liquid to further increase the solvent concentration of the mixed liquid.

As illustrated in FIG. 3, the first membrane separator 62 includes a first mixing path 62a, a first water path 62b, and a first separation membrane 62c. The mixed liquid flows into the first mixing path 62a. The first separation membrane 62c partitions the first mixing path 62a and the first water path 62b. The first separation membrane 62c is a membrane that allows water in the mixed liquid to pass therethrough and substantially blocks the organic solvent. Part of the mixed liquid flowing into the first mixing path 62a passes through the first separation membrane 62c and flows into the first water path 62b. Thus, the solvent concentration of the mixed liquid passed through the first mixing path 62a becomes higher than the solvent concentration of the mixed liquid immediately before flowing into the first mixing path 62a. The first dewaterer 60 increases the solvent concentration of the mixed liquid to greater than or equal to a predetermined recycling reference value using the first membrane separator 62. The recycling reference value is a solvent concentration usable for the processing unit 4, and, for example, is previously set. Hereinafter, the mixed liquid in which the solvent concentration is increased to greater than or equal to the recycling reference value is also referred to as a recycling liquid. It can also be said that the first dewaterer 60 separates the water from the mixed liquid from the second dewaterer 70 to generate the recycling liquid.

the upstream end of a second liquid sending pipe 85 is connected to the first dewaterer 60, and the downstream end of the second liquid sending pipe 85 is connected to a supply tank Tk3 for supply to the processing unit 4. The first dewaterer 60 supplies the recycling liquid to the supply tank Tk3 through the second liquid sending pipe 85. The mixed liquid in the supply tank Tk3 is supplied to the processing unit 4 again.

Meanwhile, the first separation membrane 62c has an application range of the solvent concentration. That is, the first separation membrane 62c can appropriately separate the water from the mixed liquid having the solvent concentration within the application range. On the other hand, when the mixed liquid having the solvent concentration less than a lower limit value of the application range flows into the first membrane separator 62, a problem may occur in first separation membrane 62c. For example, the first membrane separator 62 cannot sufficiently separate the water from the mixed liquid. Alternatively, when the ratio of water molecules passing through the first separation membrane 62c exceeds an allowable value, a crystal structure configuring the first separation membrane 62c is partially dissolved, and as a result, a service life of the first separation membrane 62c is significantly shortened. Hereinafter, the lower limit value of the application range of the solvent concentration is referred to as a concentration lower limit value. As an example, the concentration lower limit value of the first separation membrane 62c is 50 wt %.

In the first embodiment, the solvent concentration of the mixed liquid discharged from the processing unit 4 is less than the concentration lower limit value of the first separation membrane 62c. The mixed liquid is supplied to the second dewaterer 70 at the preceding stage of the first dewaterer 60. The concentration lower limit value of the second dewaterer 70 is less than or equal to the solvent concentration of the mixed liquid discharged from the processing unit 4. For this reason, the second dewaterer 70 can appropriately separate the water from the mixed liquid discharged from the processing unit 4 to increase the solvent concentration of the mixed liquid. The second dewaterer 70 increases the solvent concentration of the mixed liquid to greater than or equal to the concentration lower limit value of the first separation membrane 62c. Then, the second dewaterer 70 supplies the separated mixed liquid to the first dewaterer 60.

The solvent concentration of the mixed liquid from the second dewaterer 70 is greater than or equal to the concentration lower limit value of the first separation membrane 62c, so that the first dewaterer 60 can appropriately separate the water from the mixed liquid using the first membrane separator 62. As a result, the first dewaterer 60 can appropriately generate the recycling liquid.

As described above, the organic solvent collection part 5 increases the solvent concentration of the mixed liquid discharged from the processing unit 4 and generates the recycling liquid. This recycling liquid is again supplied to the processing unit 4. That is, the substrate processing apparatus 100 recycles the organic solvent in the mixed liquid discharged from the processing unit 4. According to this, an amount of the organic solvent to be discarded can be reduced, and the organic solvent can be more effectively used. That is, the organic solvent collection part 5 contributes to liquid saving.

Moreover, in the first embodiment, first, the second dewaterer 70 increases the solvent concentration of the mixed liquid from the processing unit 4. For this reason, even when the solvent concentration of the mixed liquid from the processing unit 4 is less than the lower limit value of the application range of the first separation membrane 62c, the second dewaterer 70 can increase the solvent concentration of the mixed liquid to greater than or equal to the concentration lower limit value of the first separation membrane 62c. Consequently, the first membrane separator 62 of the first dewaterer 60 can appropriately increase the solvent concentration of the mixed liquid. In other words, reliability of the organic solvent collection part 5 can be improved.

In addition, because the first dewaterer 60 separates the water from the mixed liquid using the first membrane separator 62, efficiency of the first dewaterer 60 is high. For example, the energy efficiency of the first dewaterer 60 is higher than the energy efficiency of other separation types such as distillation. That is, in the first embodiment, after the second dewaterer 70 increases the solvent concentration of the mixed liquid to greater than or equal to the concentration lower limit value of the first separation membrane 62c, the first dewaterer 60 further increases the solvent concentration of the mixed liquid using the highly efficient first membrane separator 62.

Consequently, the organic solvent collection part 5 can increase the solvent concentration of the mixed liquid with higher reliability and higher efficiency. In addition, the apparatus size of the first membrane separator 62 is small, so that the organic solvent collection part 5 can be implemented with a smaller size.

### <3-1. Specific Example of Organic Solvent Collection Part 5>

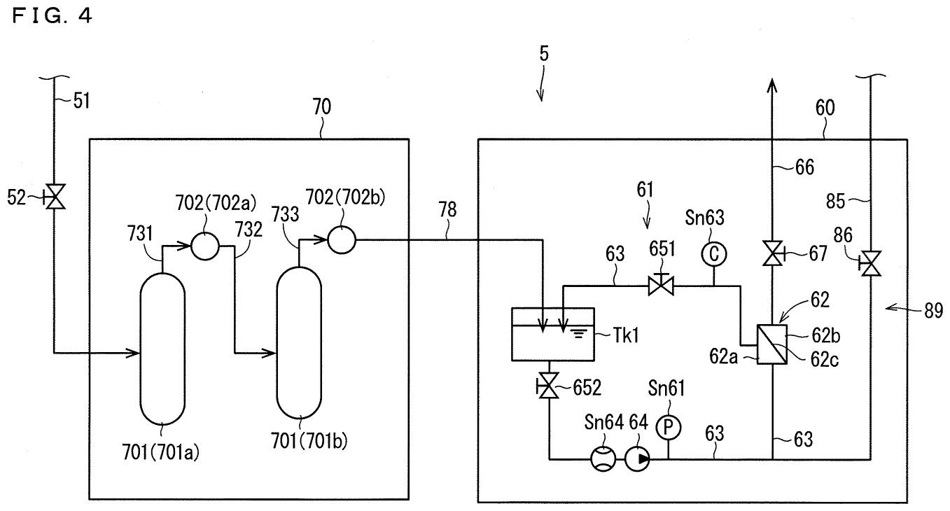

FIG. 4 is a view schematically illustrating an example of a specific configuration of the organic solvent collection part 5. The organic solvent collection part 5 can be accommodated in a first accommodation box 50a (see FIG. 1). As an example, the first accommodation box 50a is disposed outside (for example, under a clean room in which the substrate processing apparatus 100 is installed (for example, downstairs)) an outer wall 100a of the substrate processing apparatus 100.

### <3-1-1. Second Dewaterer>

In the example of FIG. 4, the second dewaterer 70 includes a distillation column 701 and a cooler 702. The downstream end of the collection pipe 51 is connected to the distillation column 701, and the upstream end of a steam pipe 731 is connected to, for example, an upper part of the distillation column 701. The downstream end of the steam pipe 731 is connected to the cooler 702.

In the example of FIG. 4, a collection valve 52 is interposed in the collection pipe 51. When the controller 6 opens the collection valve 52, the mixed liquid from the processing unit 4 is supplied to the distillation column 701 through the collection pipe 51. The distillation column 701 includes a heater (not illustrated) that heats the mixed liquid. The distillation column 701 separates the water from the mixed liquid by distillation using a difference between a boiling point of the organic solvent and a boiling point of water. At this point, as an example, the boiling point of the organic solvent is lower than the boiling point of water, and the volatility of the organic solvent is higher than the volatility of water. For example, the organic solvent is IPA. The distillation column 701 vaporizes the mixed liquid to supply steam containing a large amount of the organic solvent to the upstream end of the steam pipe 731. Although the steam flowing into the upstream end of the steam pipe 731 can contain not only the organic solvent but also water, the solvent concentration of the steam is higher than the solvent concentration immediately before the inflow of the distillation column 701. The steam flows into the cooler 702 through the steam pipe 731. The upstream end of a discharge pipe (not illustrated) that discharges the water may be connected to a lower part of the distillation column 701.

The upstream end of a liquid pipe 732 is also connected to the cooler 702. The cooler 702 cools and condenses the steam. For example, the cooler 702 may include a heat exchanger. The steam passes through the inside of the heat exchanger. The cooler 702 may include a heat pump type cooling source or a cooling source including a Peltier element that cools the heat exchanger. The steam is deprived of heat by the heat exchanger, and turns into a liquid (that is, the mixed liquid). This mixed liquid flows into the upstream end of the liquid pipe 732. The solvent concentration of this mixed liquid is higher than the solvent concentration of the mixed liquid immediately before distillation column 701.

As illustrated in FIG. 4, the second dewaterer 70 may include a plurality of distillation columns 701 and a plurality of coolers 702. In the example of FIG. 4, a set of the distillation column 701 and the cooler 702 is connected in series. In the example of FIG. 4, a distillation column 701a and a distillation column 701b are illustrated as the distillation column 701, and a cooler 702a and a cooler 702b are illustrated as the cooler 702. The downstream end of the collection pipe 51 is connected to the distillation column 701a, the steam pipe 731 connects the distillation column 701a and the cooler 702a, and the liquid pipe 732 connects the cooler 702a and the distillation column 701b. The steam from the distillation column 701a is condensed in the cooler 702a and changed into the mixed liquid, and the mixed liquid from the cooler 702a is supplied to the distillation column 701b. For example, the upstream end of the steam piping 733 is connected to the upper part of the distillation column 701b, and the downstream end of the steam piping 733 is connected to the cooler 702b. The steam of the mixed liquid from the distillation column 701b is cooled and condensed by the cooler 702b, and changed into the mixed liquid. The upstream end of the first liquid sending pipe 78 is connected to the cooler 702b, and the mixed liquid from the cooler 702b is supplied to the first dewaterer 60 through the first liquid sending pipe 78.

The second dewaterer 70 may include a pump and a valve (not illustrated). For example, a liquid sending valve may be interposed in the first liquid sending pipe 78, or a pump may be interposed in the liquid pipe 732.

As described above, in the example of FIG. 4, the second dewaterer 70 separates water from the mixed liquid using the distillation column 701 and the cooler 702 to increase the solvent concentration of the mixed liquid. In the example of FIG. 4, the second dewaterer 70 includes the plurality of distillation columns 701 and the plurality of coolers 702. According to this, the solvent concentration of the mixed liquid increases each time the mixed liquid passes through the set of the distillation column 701 and the cooler 702. For this reason, the second dewaterer 70 can increase the solvent concentration of the mixed liquid by a larger increase amount as compared with the case of using the single distillation column 701 and the single cooler 702. The increase amount of the solvent concentration in the second dewaterer 70 is previously set such that the solvent concentration after the increase is greater than or equal to the concentration lower limit value of the first separation membrane 62c. The number of distillation columns 701 and coolers 702 is previously set according to the increase amount.

### <3-1-2. First Dewaterer>

In the example of FIG. 4, the first dewaterer 60 includes a concentration tank Tk1 and a first circulator 61.

### (a) Concentration Tank Tk1

The downstream end of the first liquid sending pipe 78 is connected to the concentration tank Tk1. The mixed liquid is supplied from the second dewaterer 70 to the concentration tank Tk1 through the first liquid sending pipe 78. The concentration tank Tk1 stores the mixed liquid. As described above, the solvent concentration of the mixed liquid is higher than or equal to the concentration lower limit value of the first separation membrane 62c.

### (b) First Circulator 61

The first circulator 61 includes the first membrane separator 62 and a first circulation pipe 63. The first circulation pipe 63 is connected to the concentration tank Tk1. The first circulation pipe 63 is a pipe that returns the mixed liquid from the concentration tank Tk1 to the concentration tank Tk1. That is, the first circulation pipe 63 forms a first circulation path through which the mixed liquid stored in the concentration tank Tk1 is circulated so as to flow out from the concentration tank Tk1 and return to the concentration tank Tk1 again. In the example of FIG. 4, the upstream end of the first circulation pipe 63 is connected to a bottom part of the concentration tank Tk1, and the downstream end of the first circulation pipe 63 is connected to the upper part of the concentration tank Tk1.

The first membrane separator 62 is provided in the first circulation pipe 63. Specifically, the first mixing path 62a of the first membrane separator 62 is interposed in the first circulation pipe 63, and configures a part of the first circulation path of the first circulator 61. For this reason, the mixed liquid passes through the first mixing path 62a. Part of the mixed liquid flowing into the first mixing path 62a passes through the first separation membrane 62c and flows into the first water path 62b. By this dewatering, in the first circulation pipe 63, the solvent concentration of the mixed liquid immediately after the first membrane separator 62 becomes higher than the solvent concentration of the mixed liquid immediately before the first membrane separator 62. The first circulator 61 circulates the mixed liquid through the first circulation pipe 63, so that the mixed liquid continues to flow into the first membrane separator 62. For this reason, the first membrane separator 62 continues to separate the water from the mixed liquid. As a result, the solvent concentration of the mixed liquid during the circulation increases over time. Hereinafter, the liquid separated from the mixed liquid by the first membrane separator 62 is also referred to as a separation liquid. The separation liquid is almost water.

The first separation membrane 62c may be a zeolite membrane, an organic separation membrane, or a carbon nanotube (CNT) separation membrane. For example, the zeolite membrane has a crystal structure in which (SiO₄)⁴⁻ and (AlO₄)⁵⁻ having a tetrahedral structure are mutually connected. For example, the organic separation membrane is an organic film such as polyvinyl alcohol, chitosan, or polyimide. For example, the CNT separation membrane is a membrane that is obtained by adding carbon nanotubes to a membrane of polyamide or the like. Alternatively, a two-dimensional material may be adopted as the material of the first separation membrane 62c. The two-dimensional material is a material composed of one layer of atoms, and, for example, may be molybdenum sulfide (MoS₂) or a composite atomic layer compound of a front periodic transition metal (titanium, vanadium, or the like) and a light element (carbon or nitrogen). Alternatively, a metal organic frameworks (MOF) material or a carbon material (for example, graphene or graphene oxide) may be applied as the material of the first separation membrane 62c. At this point, a zeolite membrane is applied as the first separation membrane 62c.

The upstream end of a separation discharge pipe 66 is connected to the first water path 62b. The separated liquid is discharged to the outside (for example, a waste liquid processing part of a plant facility) through the separation discharge pipe 66. A decompression pump that decompresses the first water path 62b may be provided in the separation discharge pipe 66. As illustrated in FIG. 4, a discharge valve 67 is interposed in the separation discharge pipe 66.

In the example of FIG. 4, the first circulator 61 includes a pump 64 which is examples of the liquid sending part, a first selecting valve 651, and a second selecting valve 652, in addition to the first membrane separator 62 and the first circulation pipe 63.

The pump 64 is interposed in the first circulation pipe 63. As an example, the pump 64 is provided at a position on the upstream side of the first membrane separator 62. The first selecting valve 651 and the second selecting valve 652 are interposed in the first circulation pipe 63. The first selecting valve 651 is provided at a position on the downstream side of the first membrane separator 62. The second selecting valve 652 is provided at a position on the upstream side of the pump 64.

Various sensors can be interposed in the first circulation pipe 63. For example, a concentration sensor Sn63 that measures the concentration of the organic solvent (here, for example, IPA) in the fluid flowing through the first circulation pipe 63, a flow rate sensor (flow meter) Sn64 that measures the flow rate of the fluid flowing through the first circulation pipe 63, a pressure sensor Sn61 that measures the pressure of the fluid flowing through the first circulation pipe 63 are interposed in the first circulation pipe 63. For example, the concentration sensor Sn63 is interposed at a position on the downstream side of the first membrane separator 62. For example, the flow rate sensor Sn64 is interposed at a position on the upstream side of the pump 64. For example, the pressure sensor Sn61 is interposed at a position on the downstream side of the pump 64 and the upstream side of the first membrane separator 62.

### <3-1-3. Recycling Liquid Supply Part 89>

In the example of FIG. 4, the first dewaterer 60 also includes a recycling liquid supply part 89. The recycling liquid supply part 89 supplies the recycling liquid to the supply tank Tk3. The recycling liquid supply part 89 includes the second liquid sending pipe 85, a liquid sending valve 86, and the pump 64 as an example of the liquid sending part.

In the example of FIG. 4, the supply tank Tk3 is connected to the first circulation pipe 63 through the second liquid sending pipe 85. That is, the downstream end of the second liquid sending pipe 85 is connected to the supply tank Tk3, and the upstream end of the second liquid sending pipe 85 is connected to the first circulation pipe 63. Specifically, the upstream end of the second liquid sending pipe 85 is connected to the first circulation pipe 63 at a position between the pump 64 and the first selecting valve 651. As an example, the upstream end of the second liquid sending pipe 85 is connected to the first circulation pipe 63 at a position between the pump 64 and the first membrane separator 62. The liquid sending valve 86 is interposed in the second liquid sending pipe 85. The upstream end of the second liquid sending pipe 85 is not necessarily connected to the first circulation pipe 63, but may be connected to the concentration tank Tk1. In this case, a pump different from the pump 64 is provided in the second liquid sending pipe 85.

### <New Liquid Supply>

As illustrated in FIG. 3, the supply tank Tk3 is connected to a new liquid supply source 403 through a new liquid pipe 401. That is, the downstream end of the new liquid pipe 401 is connected to the supply tank Tk3, and the upstream end of the new liquid pipe 401 is connected to the new liquid supply source 403. The new liquid supply source 403 is a supply source of an unused organic solvent (for example, IPA having a concentration greater than or equal to 99.8 wt %) that has never been supplied to the substrate W. A new liquid valve 402 is interposed in the new liquid pipe 401.

The supply tank Tk3 is connected to the IPA nozzle 43c through a third liquid sending pipe 404. That is, the supply tank Tk3 is connected to one end side of the third liquid sending pipe 404, and the IPA nozzle 43c (specifically, the IPA pipe 432c connected to the IPA nozzle 43c) is connected to the other end side of the third liquid sending pipe 404. At this point, for example, the third liquid sending pipe 404 is connected to the IPA nozzle 43c included in each of the plurality of processing units 4 belonging to the same tower.

A pump (supply-side liquid sending pump) 405 is interposed in the third liquid sending pipe 404. In the third liquid sending pipe 404, a filter 407 is interposed at a position on the downstream side of the supply-side liquid sending pump 405. In the third liquid sending pipe 404, a temperature regulator 406 is interposed at a position on the upstream side of the filter 407 and on the downstream side of the supply-side liquid sending pump 405.

Various sensors are interposed in the third liquid sending pipe 404. For example, a temperature sensor Sn41 that measures the temperature of the fluid flowing through the third liquid sending pipe 404 is interposed in the third liquid sending pipe 404. For example, the temperature sensor Sn41 is interposed at a position on the upstream side of the filter 407 and on the downstream side of the temperature regulator 406. The temperature regulator 406 adjusts the temperature of the recycling liquid supplied to the processing unit 4 to a predetermined temperature range corresponding to the processing of the substrate W.

### <3-2. Example of Operation of Organic Solvent Collection Part 5>

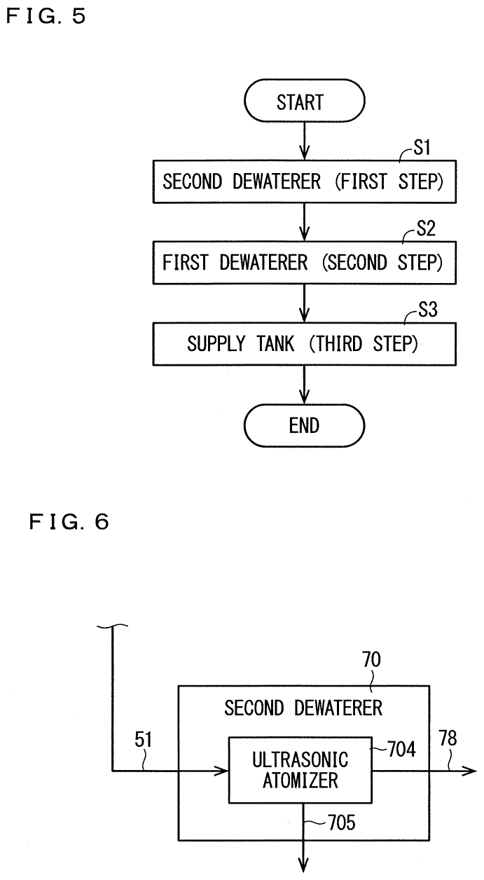

FIG. 5 is a flowchart illustrating an example of the operation of the organic solvent collection part 5. First, the second dewaterer 70 separates the water from the mixed liquid from the processing unit 4 to increase the solvent concentration of the mixed liquid (step S1: second dewaterer step: corresponding to first step). Specifically, first, the controller 6 opens the collection valve 52 to supply the mixed liquid from the processing unit 4 to the second dewaterer 70 through the collection pipe 51. The controller 6 controls the distillation column 701 and the cooler 702 to cause the second dewaterer 70 to increase the solvent concentration of the mixed liquid to greater than or equal to the concentration lower limit value (for example, 50 wt %) of the first separation membrane 62c. As an example, the solvent concentration of the mixed liquid after the separation by the second dewaterer 70 is a value (for example, about 60%) closer to the concentration lower limit value of the first separation membrane 62c (for example, 50 wt %) than the recycling reference value (for example, 99 wt %). This mixed liquid is supplied to the first dewaterer 60 through the first liquid sending pipe 78. As an example, the mixed liquid is stored in the concentration tank Tk1.

Subsequently, the first dewaterer 60 separates the water from the mixed liquid to further increase the solvent concentration of the mixed liquid (step S2: first dewaterer step: corresponding to second step). Specifically, the first circulator 61 circulates the mixed liquid through the first circulation pipe 63. As an example, the controller 6 opens the first selecting valve 651, the second selecting valve 652, and the discharge valve 67 and operates the pump 64. Thus, the mixed liquid circulates through the first circulation path including the concentration tank Tk1 and the first circulation pipe 63. By this circulation, the mixed liquid continues to pass through the first membrane separator 62. For this reason, the first membrane separator 62 continues to separate the separated liquid from the mixed liquid, and the separated liquid continues to be discharged to the outside through the separation discharge pipe 66. Consequently, the solvent concentration of the mixed liquid during the circulation increases over time.

The controller 6 causes the first circulator 61 to circulate the mixed liquid until the solvent concentration of the mixed liquid during the circulation becomes greater than or equal to the predetermined recycling reference value. For example, the recycling reference value may be greater than or equal to 60 wt %, greater than or equal to 70 wt %, greater than or equal to 80 wt %, greater than or equal to 90 wt %, greater than or equal to 95 wt %, or greater than or equal to 99 wt %. For example, the controller 6 may compare the solvent concentration measured by the concentration sensor Sn63 with the recycling reference value, and may cause the first circulator 61 to stop the circulation when the solvent concentration becomes greater than or equal to the recycling reference value. Specifically, the controller 6 closes the first selecting valve 651, the second selecting valve 652, and the discharge valve 67 and stops the pump 64.

By this circulation, the mixed liquid (that is, the recycling liquid) having an increased solvent concentration is stored in the concentration tank Tk1. The controller 6 may cause the first circulator 61 to stop the circulation with the elapse of a predetermined first dewatering time as a trigger. For example, the first dewatering time is previously set to greater than or equal to a time required for the solvent concentration to become greater than or equal to the recycling reference value. For example, the first dewatering time can be set to greater than or equal to several tens of minutes or greater than or equal to several hours.

Subsequently, the recycling liquid supply part 89 supplies the recycling liquid to the supply tank Tk3 (step S3: supply step: third step). Specifically, the controller 6 opens the liquid sending valve 86 and operates the pump 64. Thus, the recycling liquid in the concentration tank Tk1 is supplied to the supply tank Tk3 through at least the second liquid sending pipe 85.

As described above, the organic solvent collection part 5 increases the solvent concentration of the mixed liquid from the processing unit 4, and supplies the mixed liquid as the recycling liquid to the supply tank Tk3.

In the above example, first, the second dewaterer 70 increases the solvent concentration of the mixed liquid using the distillation column 701 and the cooler 702 (step S1). The concentration lower limit value of the distillation column 701 may be very low, for example, approximately zero. For this reason, even when the solvent concentration of the mixed liquid discharged from the processing unit 4 is very low, the second dewaterer 70 can appropriately increase the solvent concentration of the mixed liquid. On the other hand, power consumption of the distillation column 701 and the cooler 702 is relatively large, and energy efficiency is low. For example, the energy efficiency referred to herein may be a ratio of an increase amount of the solvent concentration to electric power. In addition, an apparatus size of the distillation column 701 is larger than that of the first membrane separator 62. In addition, due to a principle of the distillation, it is difficult for the second dewaterer 70 to increase the solvent concentration of the mixed liquid to greater than or equal to an azeotropic point.

In the first embodiment, after the second dewaterer 70 increases the solvent concentration of the mixed liquid to greater than or equal to the concentration lower limit value of the first separation membrane 62c, the first dewaterer 60 further increases the solvent concentration of the mixed liquid using the first membrane separator 62 (step S2). The energy efficiency of the membrane separation is higher than the energy efficiency of the distillation, so that the organic solvent collection part 5 can increase the solvent concentration of the mixed liquid with higher efficiency. That is, the organic solvent collection part 5 can increase the solvent concentration of the mixed liquid with higher efficiency as compared with the case where only the second dewaterer 70 increases the solvent concentration of the mixed liquid. Moreover, according to the principle of the membrane separation by the first membrane separator 62, the solvent concentration of the mixed liquid can also be increased to greater than or equal to the azeotropic point. That is, the organic solvent collection part 5 can more easily increase the solvent concentration of the mixed liquid to a value higher than or equal to the azeotropic point.

In the above example, the first dewaterer 60 causes the mixed liquid to repeatedly flow into the first membrane separator 62 by the circulation with the first circulator 61 to increase the solvent concentration of the mixed liquid. The increase amount of the solvent concentration by the first membrane separator 62 increases as the size (that is, the size of the first separation membrane 62c) of the first membrane separator 62 increases. For this reason, when the first dewaterer 60 does not circulate the mixed liquid, it is necessary to increase the size of the first membrane separator 62 in order to secure the increase amount of the solvent concentration. On the other hand, in the above-described specific example, the first dewaterer 60 increases the solvent concentration of the mixed liquid by the circulation with the first circulator 61. For this reason, the size (that is, the size of the first separation membrane 62c) of the first membrane separator 62 required to increase the solvent concentration of the mixed liquid to the recycling reference value can be reduced.

### Second Embodiment

A configuration of a substrate processing apparatus 100 according to a second embodiment is similar to the substrate processing apparatus 100 of the first embodiment. However, in the second embodiment, a specific example of the second dewaterer 70 is different from that of the first embodiment. FIG. 6 is a view schematically illustrating an example of the second dewaterer 70 according to the second embodiment. In the example of FIG. 6, the second dewaterer 70 includes an ultrasonic atomizer 704. The downstream end of the collection pipe 51, the upstream end of the first liquid sending pipe 78, and the upstream end of a separation discharge pipe 705 are connected to the ultrasonic atomizer 704.

The mixed liquid flows into the ultrasonic atomizer 704 through the collection pipe 51. The ultrasonic atomizer 704 makes the mixed liquid into mist by ultrasonic vibration. The mist of the mixed liquid includes mist of the organic solvent and mist of water. The mass distributions of these pieces of mist are different from each other. For example, the mist of the organic solvent tends to be lighter than the mist of water. The ultrasonic atomizer 704 mainly moves upward the mist of the light organic solvent and mainly moves downward the mist of heavy water, and separates the water from the mixed liquid.

For example, the ultrasonic atomizer 704 includes an atomization tank, an ultrasonic transducer, a separation container, and a gas supply part, all of which are not illustrated. The mixed liquid from the collection pipe 51 flows into the atomization tank. The ultrasonic transducer atomizes the mixed liquid in the tank. The mist from the atomization tank flows into the separation container. The mist includes the mist of the organic solvent and the mist of water. The gas supply part supplies gas from the lower part of the separation container, and mainly moves the mist of heavy water downward while mainly moving the mist of the light organic solvent upward. The upstream end of the separation discharge pipe 705 is connected to the lower part of the separation container. For this reason, the mist of the water from the separation container mainly flows into the separation discharge pipe 705. The upstream end of the first liquid sending pipe 78 is connected to the upper part of the separation container. The mist of the organic solvent is supplied to the first dewaterer 60 through the first liquid sending pipe 78. A tank that joins the mist of the organic solvent may be provided between the separation container and the first liquid sending pipe 78.

The concentration lower limit value of the ultrasonic atomizer 704 is also very low. For example, the concentration lower limit value is substantially zero. For this reason, even when the solvent concentration of the mixed liquid discharged from the processing unit 4 is very low, the second dewaterer 70 can appropriately increase the solvent concentration of the mixed liquid.

On the other hand, the ultrasonic atomizer 704 requires electric power that vibrates the ultrasonic vibrator and electric power that supplies gas. In addition, when gas other than air (for example, nitrogen gas or rare gas) is adopted as the gas, the cost of the gas is also required, and the running cost increases.

In the first embodiment, after the second dewaterer 70 increases the solvent concentration of the mixed liquid to greater than or equal to the concentration lower limit value of the first separation membrane 62c, the first dewaterer 60 further increases the solvent concentration of the mixed liquid using the first membrane separator 62. The energy efficiency of the membrane separation is higher than the energy efficiency of the atomization separation, so that the organic solvent collection part 5 can increase the solvent concentration of the mixed liquid with higher efficiency. For this reason, the organic solvent collection part 5 can increase the solvent concentration of the mixed liquid with higher efficiency. In addition, the running cost can be reduced.

### Third Embodiment

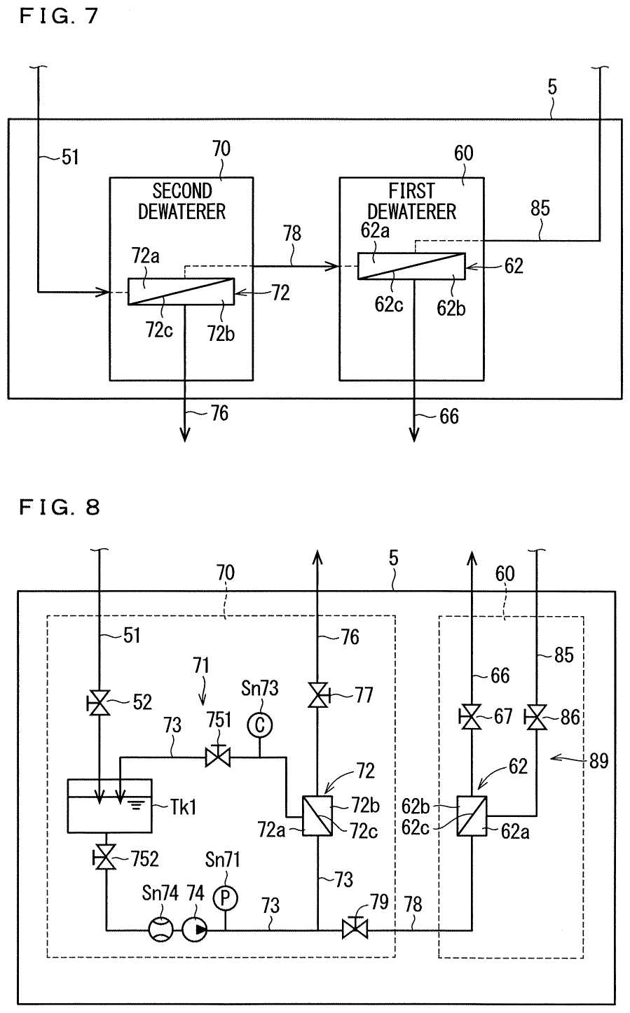

FIG. 7 is a view schematically illustrating an example of an organic solvent collection part 5 according to a third embodiment. In the example of FIG. 7, the second dewaterer 70 includes a second membrane separator 72. The mixed liquid discharged from the processing unit 4 flows into the second membrane separator 72. The second membrane separator 72 separates the water from the mixed liquid to increase the solvent concentration of the mixed liquid. The second membrane separator 72 includes a second mixing path 72a, a second water path 72b, and a second separation membrane 72c. The second mixing path 72a, the second water path 72b, and the second separation membrane 72c are similar to the first mixing path 62a, the first water path 62b, and the first separation membrane 62c, respectively.

Part of the water in the mixed liquid flowing into the second mixing path 72a passes through the second separation membrane 72c and flows into the second water path 72b. The separated liquid flowing into the second water path 72b is discharged to the outside (for example, the waste liquid processing part of the plant facility) through the separation discharge pipe 76.

The solvent concentration of the mixed liquid passing through the second mixing path 72a is higher than the solvent concentration of the mixed liquid immediately before flowing into the second mixing path 72a. The second dewaterer 70 increases the solvent concentration of the mixed liquid to greater than or equal to the concentration lower limit value of the first separation membrane 62c using the second membrane separator 72.

The concentration lower limit value of the second separation membrane 72c is lower than the concentration lower limit value of the first separation membrane 62c, and is lower than or equal to the solvent concentration of the mixed liquid from the processing unit 4. Here, the case where the first separation membrane 62c and the second separation membrane 72c are zeolite membranes will be described. The concentration lower limit value of the zeolite membrane is caused by a difference in a lattice structure of the zeolite membrane. The difference in the lattice structure of the zeolite membrane can be indicated by a type (also referred to as a structural code). Examples of the type of the zeolite membrane include an LTA type, a CHA type, and a DDR type. For example, the concentration lower limit value of the LTA type zeolite membrane is about 50 wt %, the concentration lower limit value of the CHA type zeolite membrane is about 70 wt %, and the concentration lower limit value of the DDR type zeolite membrane is about 90 wt %.

As an example, the second separation membrane 72c is the LTA type zeolite membrane, and the first separation membrane 62c is the CHA type or DDR type zeolite membrane. As another example, the second separation membrane 72c is the CHA type zeolite membrane, and the first separation membrane 62c is the DDR type zeolite membrane. More generally, the first separation membrane 62c is a first type zeolite membrane, and the second separation membrane 72c is a second type zeolite membrane having the concentration lower limit value lower than that of the first type zeolite membrane.

At this point, it is also assumed that the solvent concentration of the mixed liquid from the processing unit 4 becomes less than the concentration lower limit value of the second separation membrane 72c. In this case, a third dewaterer (not illustrated) may be provided at the preceding stage of the second dewaterer 70. The third dewaterer separates the water from the mixed liquid from the processing unit 4 to increase the solvent concentration of the mixed liquid to greater than or equal to the concentration lower limit value of the second separation membrane 72c. The third dewaterer supplies the separated mixed liquid to the second dewaterer 70. As an example, the third dewaterer may include the distillation column 701 and the cooler 702, and may include the ultrasonic atomizer 704.

In addition, a separation constant of the first separation membrane 62c is higher than a separation constant of the second separation membrane 72c. The separation constant referred to herein is an index indicating the solvent concentration of the mixed liquid after the mixed liquid is circulated under predetermined constant conditions in the circulation path in which the membrane separator is provided. For example, the conditions here include an initial value of the solvent concentration of the mixed liquid, a flow rate and a temperature during the circulation of the mixed liquid, and a circulation time. The higher the solvent concentration of the mixed liquid after the circulation, the larger the separation constant. Conversely, the higher the separation constant, the more the membrane separator can increase the solvent concentration of the organic solvent by the greater increase amount.

As described above, in the third embodiment, the second dewaterer 70 includes the second membrane separator 72. For this reason, the second dewaterer 70 can separate the water from the mixed liquid with high efficiency to increase the solvent concentration of the mixed liquid as compared with a method such as distillation and atomization separation.

The concentration lower limit value of the second separation membrane 72c is lower than the concentration lower limit value of the first separation membrane 62c. For this reason, even when the solvent concentration of the mixed liquid from the processing unit 4 is less than the concentration lower limit value of the first separation membrane 62c, the organic solvent collection part 5 can increase the solvent concentration of the mixed liquid to greater than or equal to the recycling reference value as long as the solvent concentration is greater than or equal to the concentration lower limit value of the second separation membrane 72c. Moreover, the separation constant of the first separation membrane 62c is larger than the separation constant of the second separation membrane 72c. For this reason, the organic solvent collection part 5 can increase the solvent concentration of the mixed liquid to greater than or equal to the recycling reference value with higher efficiency as compared with the case where only the second dewaterer 70 increases the solvent concentration of the mixed liquid.

### <First More Specific Example of Organic Solvent Collection Part 5>

FIG. 8 is a view schematically illustrating a first example of a more specific configuration of the organic solvent collection part 5 according to the third embodiment.

In the example of FIG. 8, the second dewaterer 70 includes the concentration tank Tk1 and a second circulator 71. The downstream end of the collection pipe 51 is connected to the concentration tank Tk1. The concentration tank Tk1 stores the mixed liquid flowing in from the processing unit 4 through the collection pipe 51. At this point, it is assumed that the solvent concentration of the mixed liquid from the processing unit 4 is greater than or equal to the concentration lower limit value (for example, 50 wt %) of the second separation membrane 72c and less than the concentration lower limit value (for example, 70 wt %) of the first separation membrane 62c.

The second circulator 71 includes the second membrane separator 72 and a second circulation pipe 73. The second circulation pipe 73 is connected to the concentration tank Tk1. The second circulation pipe 73 is a pipe that returns the mixed liquid from the concentration tank Tk1 to the concentration tank Tk1. That is, the second circulation pipe 73 forms a second circulation path through which the mixed liquid stored in the concentration tank Tk1 flows out from the concentration tank Tk1 and returns to the concentration tank Tk1 again. In the example of FIG. 8, the upstream end of the second circulation pipe 73 is connected to the bottom part of the concentration tank Tk1, and the downstream end of the second circulation pipe 73 is connected to the upper part of the concentration tank Tk1.

The second membrane separator 72 is provided in the second circulation pipe 73. Specifically, the second mixing path 72a of the second membrane separator 72 is interposed in the second circulation pipe 73, and configures a part of the second circulation path of the second circulator 71. For this reason, the mixed liquid passes through the second mixing path 72a. Part of the water in the mixed liquid flowing into the second mixing path 72a passes through the second separation membrane 72c and flows into the second water path 72b. By this dewatering, in the second circulation pipe 73, the solvent concentration of the mixed liquid immediately after the second membrane separator 72 becomes higher than the solvent concentration of the mixed liquid immediately before the second membrane separator 72. Because the second circulator 71 circulates the mixed liquid through the second circulation pipe 73, the mixed liquid continues to flow into the second membrane separator 72. For this reason, the second membrane separator 72 continues to separate the water from the mixed liquid. As a result, the solvent concentration of the mixed liquid during the circulation increases over time.

The upstream end of the separation discharge pipe 76 is connected to the second water path 72b. The separated liquid is discharged to the outside (for example, the waste liquid processing part of the plant facility) through the separation discharge pipe 76. A decompression pump that decompresses the second water path 72b may be provided in the separation discharge pipe 76. As illustrated in FIG. 8, a discharge valve 77 is interposed in the separation discharge pipe 76.

In the example of FIG. 8, the second circulator 71 includes a pump 74, a first selecting valve 751, and a second selecting valve 752, which are examples of the liquid sending parts, in addition to the second membrane separator 72 and the second circulation pipe 73. The pump 74, the first selecting valve 751, and the second selecting valve 752 are similar to the pump 64, the first selecting valve 651, and the second selecting valve 652 in the first embodiment, respectively.

Various sensors can be interposed in the second circulation pipe 73. For example, a concentration sensor Sn73, a flow rate sensor Sn74, and a pressure sensor Sn71 are interposed in the second circulation pipe 73. The concentration sensor Sn73, the flow rate sensor Sn74, and the pressure sensor Sn71 are similar to the concentration sensor Sn63, the flow rate sensor Sn64, and the pressure sensor Sn61 in the first embodiment, respectively.

In the example of FIG. 8, the upstream end of the first liquid sending pipe 78 is connected to the second circulation pipe 73 at a position between the pump 74 and the first selecting valve 751. As an example, the upstream end of the first liquid sending pipe 78 is connected to the second circulation pipe 73 at a position between the pump 74 and the second membrane separator 72. The upstream end of the first liquid sending pipe 78 is not necessarily connected to the second circulation pipe 73, but may be connected to the concentration tank Tk1. In this case, a pump different from the pump 74 is provided in the first liquid sending pipe 78. In the example of FIG. 8, a liquid sending valve 79 is interposed in the first liquid sending pipe 78.

As described above, the second dewaterer 70 includes the second circulator 71. The second circulator 71 circulates the mixed liquid through a second circulation path including the concentration tank Tk1 and the second circulation pipe 73. Specifically, the controller 6 opens the first selecting valve 751, the second selecting valve 752, and the discharge valve 77 to operate the pump 74. Thus, the mixed liquid continues to flow into second membrane separator 72. That is, the mixed liquid passes through the second membrane separator 72 repeatedly flows into the second membrane separator 72 through the second circulation path. Thus, the solvent concentration of the mixed liquid in the concentration tank Tk1 increases over time.

The controller 6 circulates the mixed liquid by the second circulator 71 until the solvent concentration of the mixed liquid in circulation reaches at least greater than or equal to the concentration lower limit value of first separation membrane 62c. For example, the controller 6 may compare the solvent concentration measured by the concentration sensor Sn73 with a concentration reference value, and may cause the second circulator 71 to stop the circulation when the solvent concentration becomes greater than or equal to the concentration reference value. Specifically, the controller 6 closes the first selecting valve 751, the second selecting valve 752, and the discharge valve 77 to stop the pump 74. Thus, the mixed liquid having the solvent concentration greater than or equal to the concentration reference value is stored in the concentration tank Tk1. The concentration reference value can previously be set to a value greater than or equal to the concentration lower limit value (for example, 70 wt %) of the first separation membrane 62c. For example, the concentration reference value may be set to a value closer to the concentration lower limit value (for example, 99 wt %) of the first separation membrane 62c than the recycling reference value (for example, 70 wt %).

The controller 6 may cause the second circulator 71 to stop the circulation with the elapse of a predetermined second dewatering time as a trigger. For example, the second dewatering time is previously set to greater than or equal to a time required for the solvent concentration to become greater than or equal to the concentration reference value. For example, the second dewatering time can be set to greater than or equal to several tens of minutes or greater than or equal to several hours.

As described above, according to the organic solvent collection part 5 according to the first example, the solvent concentration of the mixed liquid is increased by the circulation with the second circulator 71. That is, the mixed liquid repeatedly flows into the second membrane separator 72. For this reason, the size (that is, the size of the second separation membrane 72c) of the second membrane separator 72 required to increase the solvent concentration of the mixed liquid to the concentration reference value can be reduced.

### <First Dewaterer>

In the example of FIG. 8, the downstream end of the first liquid sending pipe 78 is connected to the first membrane separator 62 of the first dewaterer 60. Specifically, the downstream end of the first liquid sending pipe 78 is connected to an upstream portion of the first mixing path 62a of the first membrane separator 62. The upstream end of the second liquid sending pipe 85 is connected to a downstream portion of the first mixing path 62a of the first membrane separator 62. The upstream end of the separation discharge pipe 66 is connected to the first water path 62b of the first membrane separator 62.

In the first dewaterer 60, the controller 6 opens the second selecting valve 752, the liquid sending valve 79, the discharge valve 67, and the liquid sending valve 86 to operate the pump 74. Thus, the mixed liquid from the second dewaterer 70 flows into the first membrane separator 62 through the first liquid sending pipe 78. The first membrane separator 62 separates the water from the mixed liquid, and supplies the separated mixed liquid (that is, the recycling liquid) to the supply tank Tk3 through the second liquid sending pipe 85 while discharging the separated liquid to the outside through the separation discharge pipe 66.

In the first example, the first dewaterer 60 does not circulate the mixed liquid, so that the time required for the operation by the first dewaterer 60 can be shortened.

An example of the operation of the organic solvent collection part 5 according to the first example is similar to that in FIG. 5. That is, the second dewaterer 70 increases the solvent concentration of the mixed liquid to greater than or equal to the concentration lower limit value of the first separation membrane 62c as described above (step S1), the first dewaterer 60 increases the solvent concentration of the mixed liquid from the second dewaterer 70 to greater than or equal to the recycling reference value as described above (step S2), and the recycling liquid supply part 89 supplies the mixed liquid (recycling liquid) to the supply tank Tk3 (step S3).

### <Second More Specific Example of Organic Solvent Collection Part>

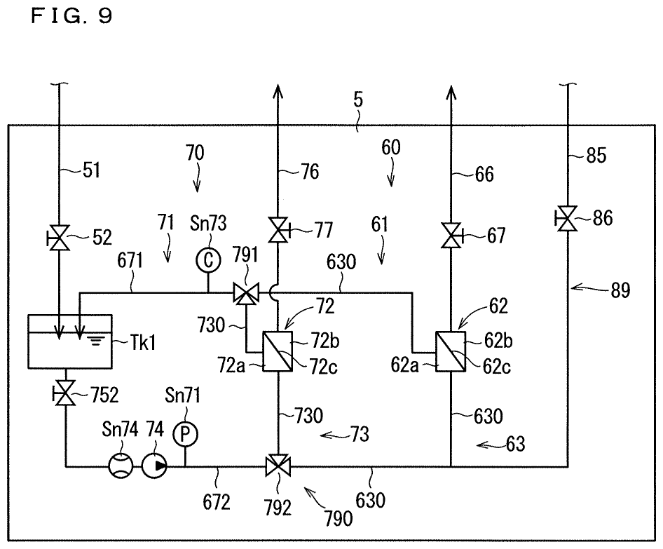

FIG. 9 is a view schematically illustrating a second example of the more specific configuration of the organic solvent collection part 5 according to the third embodiment. The organic solvent collection part 5 according to the second example is different from the organic solvent collection part 5 according to the first example in the configuration of the first dewaterer 60. The first dewaterer 60 according to the second example has substantially the same configuration as the first dewaterer 60 in FIG. 4. However, in the example of FIG. 9, a part of the first circulation pipe 63 is shared by a part of the second circulation pipe 73.

In the example of FIG. 9, the first circulation pipe 63 includes a downstream common pipe 671, a first individual pipe 630, and an upstream common pipe 672 that is an example of the common circulation pipe, and the second circulation pipe 73 includes the downstream common pipe 671, a second individual pipe 730, and an upstream common pipe 672. That is, the downstream common pipe 671 and the upstream common pipe 672 are shared by the first circulation pipe 63 and the second circulation pipe 73. For example, the upstream end of the upstream common pipe 672 is connected to a bottom part of the concentration tank Tk1, and the downstream end of the downstream common pipe 671 is connected to the upper part of the concentration tank Tk1. The upstream ends of the first individual pipe 630 and the second individual pipe 730 are connected to the downstream end of the upstream common pipe 672, and the downstream ends of the first individual pipe 630 and the second individual pipe 730 are connected to the upstream end of the downstream common pipe 671.

The first membrane separator 62 is provided in the first individual pipe 630, and the second membrane separator 72 is provided in the second individual pipe 730. The pump 74 and the second selecting valve 752 are inserted into the upstream common pipe 672. For this reason, the pump 74 and the second selecting valve 752 are shared by the first circulator 61 and the second circulator 71.

A selecting valve part 790 is provided in the organic solvent collection part 5. In the example of FIG. 9, the selecting valve part 790 includes a first three-way valve 791 and a second three-way valve 792. The selecting valve part 790 selects the circulation path between the first circulation path and the second circulation path. Specifically, the selecting valve part 790 selects between a first circulation state and a second circulation state, which are described below. The first circulation state is a state in which the downstream common pipe 671 and the upstream common pipe 672 communicate with each other through the first individual pipe 630. The second circulation state is a state in which the downstream common pipe 671 and the upstream common pipe 672 communicate with each other through the second individual pipe 730. In the first circulation state, the mixed liquid circulates through the first circulation path including the concentration tank Tk1 and the first circulation pipe 63. In the second circulation state, the mixed liquid circulates through the second circulation path including the concentration tank Tk1 and the second circulation pipe 73.

In the example of FIG. 9, the first three-way valve 791 is connected to the upstream end of the downstream common pipe 671, the downstream end of the first individual pipe 630, and the downstream end of the second individual pipe 730. The first three-way valve 791 selects between a first downstream circulation state in which the downstream common pipe 671 communicates with the first individual pipe 630 and a second downstream circulation state in which the downstream common pipe 671 communicates with the second individual pipe 730. The second three-way valve 792 is connected to the downstream end of the upstream common pipe 672, the upstream end of the first individual pipe 630, and the upstream end of the second individual pipe 730. The second three-way valve 792 selects between a first upstream circulation state in which the upstream common pipe 672 communicates with the first individual pipe 630 and a second upstream circulation state in which the upstream common pipe 672 communicates with the second individual pipe 730.

When the controller 6 causes the first three-way valve 791 to choose the first downstream circulation state and causes the second three-way valve 792 to choose the first upstream circulation state, the mixed liquid circulates through the first circulation path. That is, the selecting valve part 790 chooses the first circulation state. When the controller 6 causes the first three-way valve 791 to choose the second downstream circulation state and causes the second three-way valve 792 to choose the second upstream circulation state, the mixed liquid circulates through the second circulation path. That is, the selecting valve part 790 chooses the second circulation state.

Also in the second example, first, the second dewaterer 70 operates. That is, the controller 6 causes the selecting valve part 790 to choose the second circulation state when the solvent concentration of the mixed liquid in the concentration tank Tk1 is a second value less than the concentration lower limit value of the first separation membrane 62c. For example, the solvent concentration of the mixed liquid in the concentration tank Tk1 may be measured by a concentration sensor (not illustrated). The second dewaterer 70 initially operates by choosing the second circulation state. That is, the second dewaterer 70 circulates the mixed liquid to the second circulator 71 through the second circulation path. The second dewaterer 70 circulates the mixed liquid in the second circulator 71 until the solvent concentration of the mixed liquid in the concentration tank Tk1 becomes greater than or equal to the concentration lower limit value of the first separation membrane 62c. Then, the controller 6 causes the selecting valve part 790 to choose the first circulation state when the solvent concentration of the mixed liquid is a first value greater than or equal to the concentration lower limit value. Thus, the first dewaterer 60 operates. That is, the first dewaterer 60 circulates the mixed liquid to the first circulator 61 through the first circulation path. The first dewaterer 60 circulates the mixed liquid in the first circulator 61 until the solvent concentration of the mixed liquid in the concentration tank Tk1 becomes greater than or equal to the recycling reference value. Thus, the recycling liquid is stored in the concentration tank Tk1.

In the example of FIG. 9, the upstream end of the second liquid sending pipe 85 is connected to the first circulation pipe 63 (specifically, the first individual pipe 630). For example, the controller 6 opens the second selecting valve 752 and the liquid sending valve 86, causes the first three-way valve 791 to choose the second downstream circulation state, causes the second three-way valve 792 to choose the first upstream circulation state, and operates the pump 74. Thus, the mixed liquid (recycling liquid) in the concentration tank Tk1 is supplied to the supply tank Tk3. The upstream end of the second liquid sending pipe 85 may be connected to the second circulation pipe 73 or may be connected to the concentration tank Tk1.

An example of the operation of the organic solvent collection part 5 according to the second example is similar to that in FIG. 5. However, an example of a specific operation is different. At this point, the solvent concentration of the mixed liquid from the processing unit 4 is lower than the concentration lower limit value of the first separation membrane 62c. Accordingly, first, the second dewaterer 70 increases the solvent concentration of the mixed liquid to greater than or equal to the concentration lower limit value of the first separation membrane 62c by the circulation with the second circulator 71 (step S1). Specifically, the controller 6 opens the second selecting valve 752 and the discharge valve 77, causes the selecting valve part 790 to choose the second circulation state, and operates the pump 74. Thus, the mixed liquid circulates through the second circulation path including the concentration tank Tk1 and the second circulation pipe 73. By this circulation, because the mixed liquid passes through the second membrane separator 72, the solvent concentration of the mixed liquid in the concentration tank Tk1 increases over time.

When the solvent concentration of the mixed liquid in the concentration tank Tk1 becomes greater than or equal to the concentration reference value, the second dewaterer 70 stops the circulation, and the first dewaterer 60 increases the solvent concentration of the mixed liquid to greater than or equal to the recycling reference value by the circulation with the first circulator 61 (step S2). Specifically, the controller 6 opens the discharge valve 67 and causes the selecting valve part 790 to choose the first circulation state. Thus, the mixed liquid circulates through the first circulation path including the concentration tank Tk1 and the first circulation pipe 63. By this circulation, because the mixed liquid passes through the first membrane separator 62, the solvent concentration of the mixed liquid in the concentration tank Tk1 increases over time. The controller 6 may close the discharge valve 77.

When the solvent concentration of the mixed liquid in the concentration tank Tk1 becomes greater than or equal to the recycling reference value, the first circulator 61 stops the circulation. Specifically, the controller 6 closes the second selecting valve 752 and the discharge valve 67 to stop the pump 74.

Subsequently, the recycling liquid supply part 89 supplies the mixed liquid (that is, the recycling liquid) in the concentration tank Tk1 in which the solvent concentration is increased by the first dewaterer 60 to the supply tank Tk3 (step S3).

As described above, according to the second example, the first dewaterer 60 increases the solvent concentration of the mixed liquid by the circulation with the first circulator 61. For this reason, the size (that is, the size of the first separation membrane 62c) of the first membrane separator 62 required to increase the solvent concentration of the mixed liquid to greater than or equal to the recycling reference value can be reduced.

In the example of FIG. 9, the pump 74 is shared by the first circulator 61 and the second circulator 71. For this reason, the manufacturing cost of the organic solvent collection part 5 can be reduced.

### Fourth Embodiment

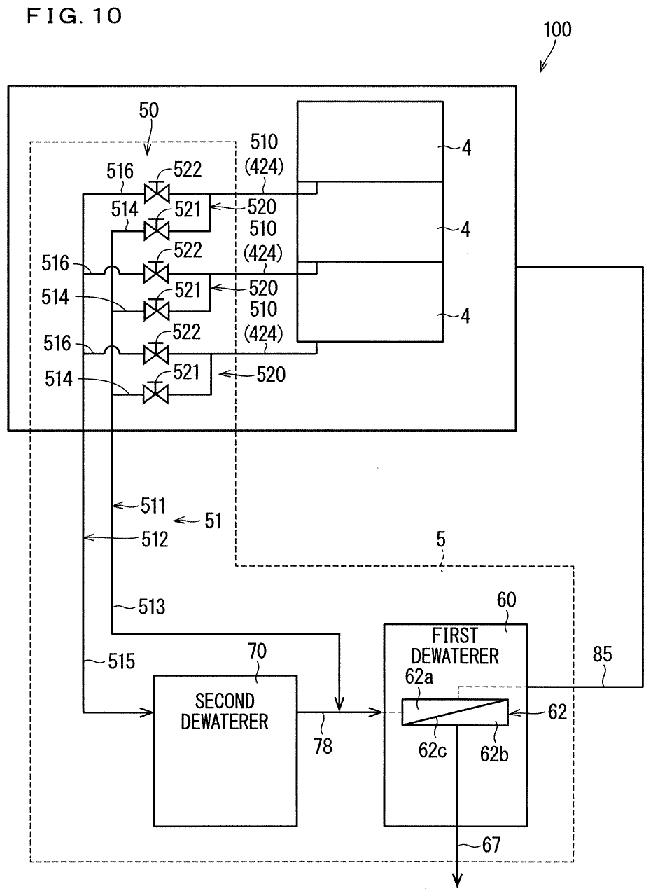

FIG. 10 is a view schematically illustrating a first example of a substrate processing apparatus 100 according to a fourth embodiment. The substrate processing apparatus 100 according to the fourth embodiment is different from the substrate processing apparatus 100 according to the first embodiment in the presence or absence of a collection destination selector 50. It can also be said that the collection destination selector 50 belongs to the organic solvent collection part 5. The collection destination selector 50 selects between a first dewatering state and a second dewatering state, which are described next. The first dewatering state is a state in which the mixed liquid from the processing unit 4 is supplied to the first dewaterer 60 while bypassing the second dewaterer 70. The second dewatering state is a state in which the mixed liquid from the processing unit 4 is supplied to the second dewaterer 70.

The collection destination selector 50 includes the collection pipe 51 and a selecting valve part 520. In the example of FIG. 10, the collection pipe 51 includes a first dewatering pipe 511, a second dewatering pipe 512, and a common collection pipe 510. In the example of FIG. 10, a plurality of common collection pipes 510 are provided corresponding to the plurality of processing units 4. In the example of FIG. 10, the plurality of common collection pipes 510 are provided on a one-to-one basis with respect to the plurality of processing units 4. The upstream end of each common collection pipe 510 is connected to the corresponding processing unit 4 (specifically, the cup 42). The common collection pipe 510 corresponds to the above-described cup-side collection pipe 424.

The downstream end of the first dewatering pipe 511 is connected to the first dewaterer 60. The first dewatering pipe 511 is connected to the downstream end of each common collection pipe 510. In the example of FIG. 10, the first dewatering pipe 511 includes a first common pipe 513 and a plurality of first branch pipes 514. The plurality of first branch pipes 514 are provided on a one-to-one basis with respect to the plurality of common collection pipes 510. The upstream end of the first branch pipe 514 is connected to the downstream end of the corresponding common collection pipe 510, and the downstream end of the first branch pipe 514 is connected to the first common pipe 513. The downstream end of the first common pipe 513 corresponds to the downstream end of the first dewatering pipe 511.

The downstream end of the second dewatering pipe 512 is connected to the second dewaterer 70. The second dewatering pipe 512 is connected to the downstream end of each common collection pipe 510. In the example of FIG. 10, the second dewatering pipe 512 includes a second common pipe 515 and a plurality of second branch pipes 516. The plurality of second branch pipes 516 are provided on a one-to-one basis with respect to the plurality of common collection pipes 510. The upstream end of the second branch pipe 516 is connected to the downstream end of the corresponding common collection pipe 510, and the downstream end of the second branch pipe 516 is connected to the second common pipe 515. The downstream end of the second common pipe 515 corresponds to the downstream end of the second dewatering pipe 512.

In the example of FIG. 10, the selecting valve part 520 includes a selecting valve 521 and a selecting valve 522. The selecting valve part 520 selects between a state in which the common collection pipe 510 communicates with the first dewaterer 60 (that is, the first dewatering state) and a state in which the common collection pipe 510 communicates with the second dewaterer 70 (that is, the second dewatering state). In the example of FIG. 10, the plurality of selecting valve parts 520 are provided on a one-to-one basis with respect to the plurality of processing units 4. That is, in the example of FIG. 10, the plurality of selecting valves 521 are provided on a one-to-one basis with respect to the plurality of processing units 4, and the plurality of selecting valves 522 is provided on a one-to-one basis with respect to the plurality of processing units 4. Each selecting valve 521 is interposed in the corresponding first branch pipe 514, and each selecting valve 522 is interposed in the corresponding second branch pipe 516.

Hereinafter, the operation of the selecting valve part 520 corresponding to one processing unit 4 will be described. When the controller 6 closes the selecting valve 521 and opens the selecting valve 522, the mixed liquid from the processing unit 4 flows through the common collection pipe 510 and the second dewatering pipe 512 in this order and is supplied to the second dewaterer 70. That is, the selecting valve part 520 selects the second dewatering state. When the controller 6 opens the selecting valve 521 and closes the selecting valve 522, the mixed liquid from the processing unit 4 flows through the common collection pipe 510 and the first dewatering pipe 511 in this order and is supplied to the first dewaterer 60. That is, the selecting valve part 520 selects the first dewatering state.

The controller 6 controls the collection destination selector 50 based on the solvent concentration of the mixed liquid discharged from the processing unit 4. Specifically, the controller 6 causes the collection destination selector 50 to choose the first dewatering state when the solvent concentration of the mixed liquid is a first value greater than or equal to the concentration lower limit value of the first separation membrane 62c, and the controller 6 causes the collection destination selector 50 to choose the second dewatering state when the solvent concentration of the mixed liquid is a second value less than the concentration lower limit value of the first separation membrane 62c. At this point, it is assumed that the concentration lower limit value of the second dewaterer 70 is substantially zero.

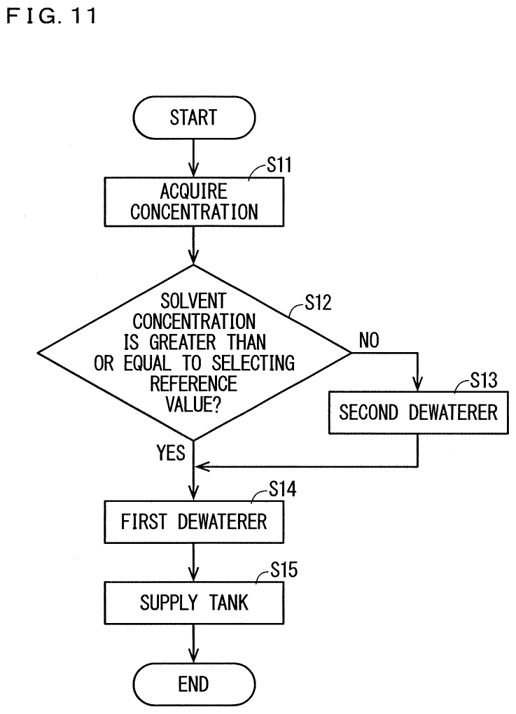

FIG. 11 is a flowchart illustrating an example of the operation of the organic solvent collection part 5 according to the fourth embodiment. First, the controller 6 acquires the solvent concentration of the mixed liquid discharged from the processing unit 4 (step S11: concentration acquisition step). At this point, the solvent concentration of the mixed liquid discharged from the processing unit 4 is the solvent concentration of the mixed liquid before the operation of the first dewaterer 60 and the second dewaterer 70. A specific example of a method for acquiring the solvent concentration will be described in detail later.

Subsequently, the controller 6 determines whether the solvent concentration is greater than or equal to a predetermined selecting reference value (step S12: concentration determination step). For example, the selecting reference value is previously set to a value greater than or equal to the concentration lower limit value of the first separation membrane 62c. For example, the selecting reference value may be a value closer to the concentration lower limit value of the first separation membrane 62c than the recycling reference value. The switching reference value may be equal to or less than the concentration reference value.

When the solvent concentration of the mixed liquid is less than the selecting reference value, the second dewaterer 70 separates the water from the mixed liquid to increase the solvent concentration of the mixed liquid (step S13: second dewaterer step). Specifically, the controller 6 causes the collection destination selector 50 to choose the second dewatering state. As an example, the controller 6 closes the selecting valve 521 and opens the selecting valve 522. Thus, the mixed liquid from the processing unit 4 is supplied to the second dewaterer 70. That is, when the solvent concentration of the mixed liquid is less than the selecting reference value, sometimes the first membrane separator 62 cannot be used, so that the organic solvent collection part 5 supplies the mixed liquid to the second dewaterer 70, and the second dewaterer 70 reliably increases the solvent concentration of the mixed liquid to greater than or equal to the concentration lower limit value of the first separation membrane 62c.

When the second dewaterer 70 increases the solvent concentration of the mixed liquid to greater than or equal to the concentration reference value, the first dewaterer 60 separates the water from the mixed liquid to further increase the solvent concentration of the mixed liquid (step S14: first dewaterer step). The first dewaterer 60 increases the solvent concentration of the mixed liquid to greater than or equal to the recycling reference value. In other words, the first dewaterer 60 generates the recycling liquid. Subsequently, the recycling liquid supply part 89 supplies the recycling liquid to the supply tank Tk3 (step S15: supply step).

On the other hand, when the solvent concentration of the mixed liquid is greater than or equal to the selecting reference value in step S12, the first dewaterer 60 separates the water from the mixed liquid discharged from the processing unit 4 to increase the solvent concentration of the mixed liquid (step S14: first dewaterer step). Specifically, the controller 6 causes the collection destination selector 50 to choose the first dewatering state. As an example, the controller 6 opens the selecting valve 521 and closes the selecting valve 522. That is, when the solvent concentration of the mixed liquid is greater than or equal to the selecting reference value, the highly efficient first membrane separator 62 can be used, so that the organic solvent collection part 5 supplies the mixed liquid from the processing unit 4 to the first dewaterer 60 while bypassing the second dewaterer 70.

The first dewaterer 60 increases the solvent concentration of the mixed liquid to greater than or equal to the recycling reference value (step S14), and the recycling liquid supply part 89 supplies the recycling liquid to the supply tank Tk3 (step S15).

As described above, in the case of the high solvent concentration of the mixed liquid from the processing unit 4, the second dewaterer 70 does not operate, and the first dewaterer 60 increases the solvent concentration of the mixed liquid. For this reason, the power consumption by the second dewaterer 70 can be avoided. On the other hand, in the case of the low solvent concentration of the mixed liquid from the processing unit 4, first, the second dewaterer 70 increases the solvent concentration of the mixed liquid to greater than or equal to the concentration lower limit value of the first separation membrane 62c. For this reason, the second dewaterer 70 can supply the mixed liquid having the solvent concentration greater than or equal to the concentration lower limit value of the first separation membrane 62c to the first dewaterer 60.

When the second dewaterer 70 includes the second membrane separator 72, the third dewaterer may be provided at the preceding stage of the second dewaterer 70. The concentration lower limit value of the third dewaterer is lower than the concentration lower limit value of the second separation membrane 72c, and, for example, may be substantially zero. The collection destination selector 50 may select the supply destination of the mixed liquid from the processing unit 4 among the first dewaterer 60, the second dewaterer 70, and the third dewaterer. For example, when the solvent concentration of the mixed liquid from the processing unit 4 is less than a second selecting reference value, the collection destination selector 50 supplies the mixed liquid to the third dewaterer. The second selecting reference value is set to be greater than or equal to the concentration lower limit value of the second separation membrane 72c and less than the concentration lower limit value of the first separation membrane 62c. The third dewaterer separates the water from the mixed liquid to increase the solvent concentration of the mixed liquid to greater than or equal to the concentration lower limit value of the second dewaterer 70. Then, the third dewaterer supplies the water to the second dewaterer 70 after the separation. When the solvent concentration of the mixed liquid from the processing unit 4 is greater than or equal to the second selecting reference value and less than the first selecting reference value, the collection destination selector 50 supplies the mixed liquid to the second dewaterer 70. The first selecting reference value is set to be greater than or equal to the concentration lower limit value of the first separation membrane 62c and less than the recycling reference value. When the solvent concentration of the mixed liquid from the processing unit 4 is greater than or equal to the first selecting reference value, the collection destination selector 50 supplies the mixed liquid to the first dewaterer 60.

### <Method for Acquiring Solvent Concentration>

### <Calculation of Solvent Concentration Based on Recipe Information>

Subsequently, an example of the method for acquiring the solvent concentration of the mixed liquid discharged from the processing unit 4 will be described. The solvent concentration of the mixed liquid discharged from the processing unit 4 depends on a processing content for the substrate W by the processing unit 4. For example, the processing unit 4 supplies the organic solvent to the substrate W after supplying the pure water to the substrate W. In this processing, when the processing unit 4 supplies the pure water to the substrate W at a large flow rate for a long time, the solvent concentration of the mixed liquid discharged from the processing unit 4 becomes relatively low. When the processing unit 4 supplies the organic solvent to the substrate W at a large flow rate for a long time, the solvent concentration of the mixed liquid discharged from the processing unit 4 becomes relatively high. As described above, the solvent concentration of the mixed liquid discharged from the processing unit 4 depends on the processing content.

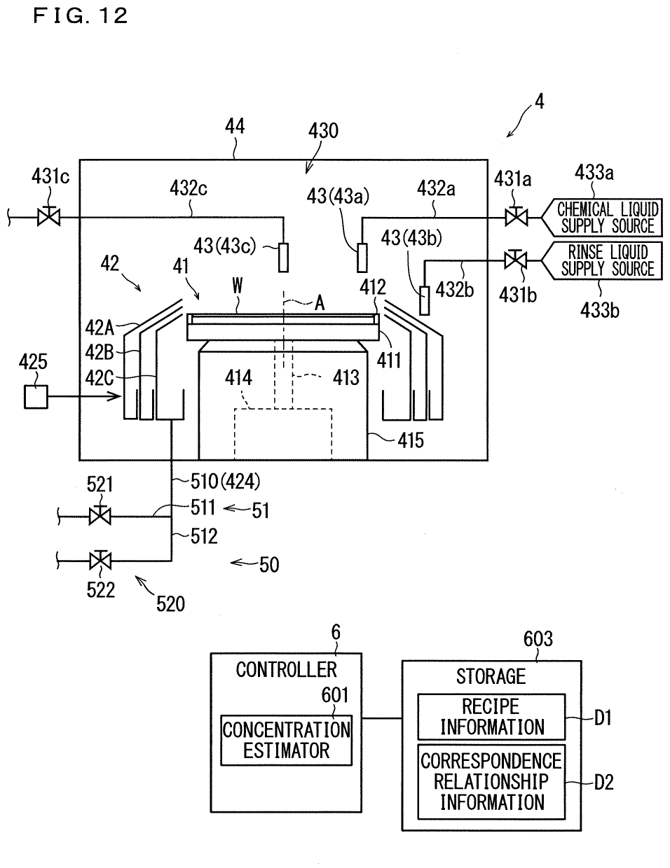

FIG. 12 is a view schematically illustrating an example of the processing unit 4 according to the fourth embodiment. As illustrated in FIG. 12, the controller 6 is connected to a storage 603. For example, the storage 603 is a nonvolatile storage, and is a memory or a hard disk as a specific example. The storage 603 stores recipe information D1 that defines the processing content for the substrates W. For example, the recipe information D1 includes various pieces of information such as a use nozzle, a flow rate of the processing liquid, a dispense time of the processing liquid, and the rotation speed of the substrate W in each piece of processing.

Also illustrated in FIG. 12, the processing unit 4 may include a plurality of cups 42. In the example of FIG. 12, a cup 42A, a cup 42B, and a cup 42C are illustrated as the plurality of cups 42. The cup 42A, the cup 42B, and the cup 42C are concentrically provided. In the example of FIG. 12, the cup 42A is located outermost, the cup 42C is located innermost, and the cup 42B is located between the cup 42A and the cup 42C.

The cup lifting mechanism 425 that lifts and lowers each cup 42. For example, the cup lifting mechanism 425 lifts the cup 42A to the upper position and lowers the cup 42B and the cup 42C to the lower positions. In this state, the processing liquid scattered from the peripheral edge of the substrate W is caught by the cup 42A. In addition, the cup lifting mechanism 425 lifts the cup 42A and the cup 42B to the upper positions and lowers the cup 42C to the lower position. In this state, the processing liquid scattered from the peripheral edge of the substrate W is caught by the cup 42B. In addition, the cup lifting mechanism 425 lifts the cup 42A, the cup 42B, and the cup 42C to the upper positions. In this state, the processing liquid scattered from the peripheral edge of the substrate W is caught by the cup 42C.

In the example of FIG. 12, the processing liquid caught by the cup 42C flows into the collection pipe 51. The processing liquid caught by the cup 42A flows into another collection pipe (not illustrated), and the processing liquid caught by the cup 42B flows into another collection pipe (not illustrated).

Such a processing unit 4 can change the cup 42 that catches the processing liquid according to the type of the processing liquid. For example, during the supply of the pure water to the substrate W, the cup lifting mechanism 425 positions only the cup 42A at the upper position. In this case, the pure water is caught by the cup 42A. During the supply of the organic solvent to the substrate W, the cup lifting mechanism 425 positions the cup 42A to the cup 42C at the upper positions. In this case, the organic solvent is caught by the cup 42C and flows into the collection pipe 51. That is, in this example, the cup 42C is the cup for the organic solvent, and the collection pipe 51 is the collection pipe for the organic solvent. As described above, the processing unit 4 can select the cup to be used between the cup 42A and the cup 42C according to the type of the processing liquid. Information indicating the position of the cup 42 in each piece of processing is also included in the recipe information D1.

As illustrated in FIG. 12, the controller 6 includes a concentration estimator 601. The concentration estimator 601 reads recipe information D1 from the storage 603. The concentration estimator 601 calculates the solvent concentration of the mixed liquid (that is, the mixed liquid flowing into the collection pipe 51) discharged from the processing unit 4 based on the recipe information D1. Table 1 is a table schematically illustrating a first example of the recipe information D1.

TABLE 1
Recipe information

| No | Rotation speed | Time | Flow rate | Processing liquid | Cup |
|---|---|---|---|---|---|
| . | . | . | . | . | . |
| . | . | . | . | . | . |
| . | . | . | . | . | . |
| 30 | 100 | 2 | 2000 | Pure water | Cup 42A |
| 31 | 10 | 1 | | | Cup 42A |
| 32 | 10 | 1 | | | Cup 42C |
| 33 | 10 | 4 | 100 | Organic solvent | Cup 42C |
| 34 | 1000 | 3 | 100 | Organic solvent | Cup 42C |
| 35 | 1000 | 2 | | | Cup 42C |
| . | . | . | . | . | . |
| . | . | . | . | . | . |
| . | . | . | . | . | . |

Table 1 illustrates some steps of the processing on the substrate W. In Table 1, the recipe information D1 includes a number of each step, the rotation speed of the substrate W in each step, the required time for each step, the flow rate of the processing liquid in each step, a type of the processing liquid in each step, and the cup used in each step. It can be said that the used cup is the information indicating the position of the cup 42.

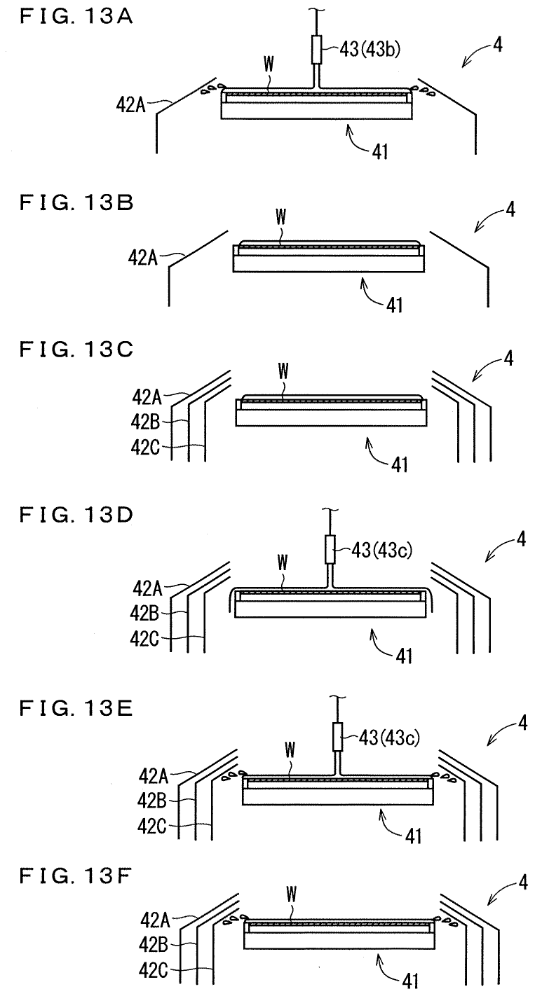

FIGS. 13A to 13F are views schematically illustrating examples of states of the processing unit 4 in respective steps of Table 1. FIGS. 13A to 13F illustrate examples of states of the processing unit 4 in a thirtieth step to a thirty-fifth step in Table 1, respectively.

In the thirtieth step in Table 1, while the spin chuck 41 rotates the substrate W at 100 rpm for 2 seconds, the rinse liquid nozzle 43b dispenses the pure water toward the substrate W at 2000 mL (milliliter)/min. In the thirtieth step, the cup 42A is used. That is, as illustrated in FIG. 13A, in the thirtieth step, the pure water scattered from the peripheral edge of the substrate W is caught by the cup 42A.

In the thirty-first step, the spin chuck 41 rotates the substrate W at 10 rpm for 1 second. In the thirty-first step, the processing liquid is not supplied to the substrate W. In the thirty-first step, because of the low rotation speed of the substrate W, the pure water is maintained on the main surface of the substrate W as illustrated in FIG. 13B. This processing is also called paddle processing. Although not illustrated in Table 1, actually, a step in which the spin chuck 41 gradually reduces the rotation speed to 10 rpm can be executed between the thirtieth step and the thirty-first step while the rinse liquid nozzle 43b dispenses the pure water at 2000 mL/min. The liquid film of the pure water on the main surface of the substrate W in the paddle processing becomes thicker as the rotation speed of the substrate W after the dispense of the pure water is stopped is lower.

In the thirty-second step, the cup lifting mechanism 425 selects the cup to be used from the cup 42A to the cup 42C while the spin chuck 41 rotates the substrate W at 10 rpm for 1 second (see FIG. 13C).

In the thirty-third step, for 4 seconds, the IPA nozzle 43c dispenses the organic solvent toward the main surface of the substrate W at 100 mL/min while the spin chuck 41 rotates the substrate W at 10 rpm. For example, the organic solvent is IPA. As illustrated in FIG. 13D, in the thirty-third step, the processing liquid (the pure water and the organic solvent) may flow down from the peripheral edge of the substrate W. In this case, the processing liquid is caught by the cup 42C and flows into the upstream end of the collection pipe 51.

In the thirty-fourth step, for 3 seconds, the IPA nozzle 43c dispenses the organic solvent toward the main surface of the substrate W at 100 mL/min while the spin chuck 41 rotates the substrate W at 1000 rpm. As illustrated in FIG. 13E, the organic solvent deposited on the main surface of the substrate W flows radially outward and scatters outward from the peripheral edge of the substrate W together with the pure water. The mixed liquid of the organic solvent and the pure water flows into the upstream end of the collection pipe 51 after being caught by the cup 42C. Through the thirty-third step and the thirty-fourth step, the pure water on the main surface of the substrate W is replaced with the organic solvent.

In the thirty-fifth step, the spin chuck 41 rotates the substrate W at 1000 rpm for 2 seconds. In the thirty-fifth step, the processing liquid is not supplied to the substrate W. As illustrated in FIG. 13F, in the thirty-fifth step, part of the organic solvent on the main surface of the substrate W scatters from the peripheral edge of the substrate W. A remaining part of the organic solvent is evaporated. For this reason, the main surface of the substrate W is dried.

As described above, the cup 42C is lifted to the upper position in thirty-first step (see also FIG. 13C). For this reason, the cup 42C can catch the processing liquid (the pure water and the organic solvent, namely, the mixed liquid) in the thirty-first step to the thirty-fifth step. The processing liquid flows into the upstream end of the collection pipe 51. Hereinafter, a period from the thirty-first step to the thirty-fifth step is also referred to as a discharge period. The discharge period is a period during which the cup 42C can catch the processing liquid.

The solvent concentration (average value) of the mixed liquid flowing into the collection pipe 51 in the discharge period can be calculated based on a pure water discharge amount and a solvent discharge amount, which are described below. The pure water discharge amount is the total amount of the pure water flowing into the collection pipe 51 in the discharge period, namely, the total amount of the pure water caught by the cup 42C in the discharge period. The solvent discharge amount is the total amount of the organic solvent flowing into the collection pipe 51 in the discharge period, namely, the total amount of the organic solvent caught by the cup 42C in the discharge period.

First, the pure water discharge amount will be described. In the example of Table 1, the pure water is not supplied during the discharge period (thirty-first step to thirty-fifth step). For this reason, the pure water discharge amount is the amount of the pure water existing on the main surface of the substrate W at the start time point of the thirty-first step (see also FIG. 13C). Hereinafter, the amount of the pure water is referred to as a pure water film amount. Because the thickness of the liquid film of the pure water existing on the main surface of the substrate W depends on the rotation speed of the substrate W at the start time point of the thirty-first step, the pure water film amount depends on the rotation speed. It can also be said that the start time point of the thirty-first step is a selecting start time point from the cup 42A to the cup 42C.

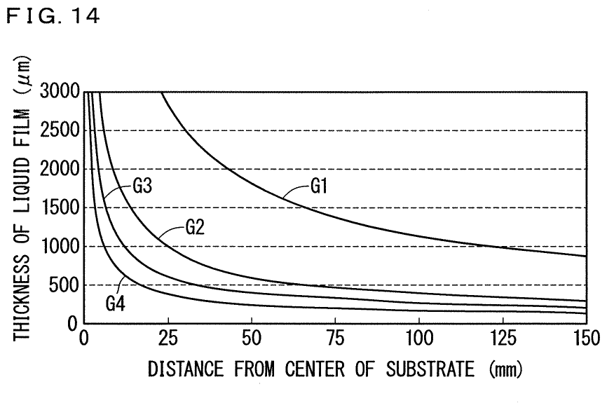

FIG. 14 is a graph illustrating an example of the distance from the center of the substrate W at each position on the substrate W and the thickness of the liquid film of the pure water at each position. That is, each graph illustrates a contour of the liquid level of the pure water. In FIG. 14, a plurality of graphs G1 to G4 having different rotation speeds of the substrate W are illustrated. The rotation speed corresponding to the graph G1 is the lowest, which is 10 rpm. The rotation speed corresponding to the graph G2 is 50 rpm, which is the second highest. The rotation speed corresponding to the graph G3 is 100 rpm, which is the third highest. The rotation speed corresponding to the graph G4 is the highest, which is 200 rpm. These graphs G1 to G4 can be obtained by simulation or an experiment.

The pure water amount (pure water film amount) existing on the main surface of the substrate W can be obtained by integrating the thickness of the liquid film of each graph. For this reason, the correspondence relationship between the rotation speed of the substrate W and the pure water film amount can previously be obtained. Correspondence relationship information D2 indicating the correspondence relationship is stored in the storage 603 (see also FIG. 12). The rotation speed at the start time point of the thirty-first step is included in the recipe information D1, so that the pure water film amount can be obtained based on the rotation speed and the correspondence relationship information D2.

The graph can also depend on the flow rate of the pure water in the thirtieth step before the paddle processing. Accordingly, a graph may previously be obtained for each flow rate by the simulation or the experiment, and the pure water film amount may be obtained from the graph. In this case, the correspondence relationship information D2 includes a correspondence relationship between the combination of the rotation speed and the pure water flow rate and the pure water film amount.

Subsequently, the solvent discharge amount will be described. For simplicity, the solvent discharge amount can be considered to be equal to the dispense amount of the organic solvent supplied to the substrate W in the discharge period. The dispense amount of the organic solvent in the discharge period can be determined by a time integral value of the solvent flow rate of the organic solvent. That is, the solvent discharge amount can be obtained by the sum of products of the solvent flow rate and the required time (dispense time) of the organic solvent in each step. In the example of Table 1, the solvent discharge amount is represented by 100×(4+3)/60. Because the organic solvent can evaporate, the solvent discharge amount may be calculated by reducing the time integral value by a predetermined ratio in view of the evaporation.

Table 2 is a table schematically illustrating a second example of the recipe information D1.

TABLE 2
Recipe information

| No | Rotation speed | Time | Flow rate | Processing liquid | Cup |
|---|---|---|---|---|---|
| . | . | . | . | . | . |
| . | . | . | . | . | . |
| . | . | . | . | . | . |
| 30 | 1500 | 4 | 2000 | Pure water | Cup 42A |
| 31 | 1500 | 2 | 2000 | Pure water | Cup 42C |
| 32 | 1500 | 0.2 | 2000/250 | Pure water/ | Cup 42C |
| | | | | organic solvent | |
| 33 | 1500 | 30 | 250 | Organic solvent | Cup 42C |
| 34 | 1500 | 30 | | | Cup 42C |
| . | . | . | . | . | . |
| . | . | . | . | . | . |
| . | . | . | . | . | . |

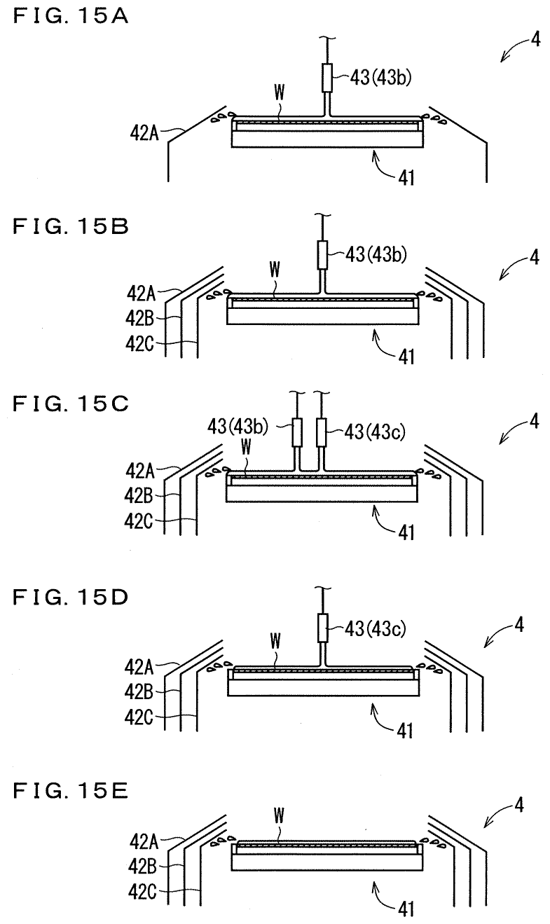

Table 2 also illustrates some steps of the processing on the substrate W. FIGS. 15A to 15E are views schematically illustrating examples of states of the processing unit 4 in respective steps of Table 2. FIGS. 15A to 15E illustrate examples of states of the processing unit 4 in the thirtieth step to the thirty-fourth step in Table 2, respectively.

In the thirtieth step in Table 2, for 4 seconds, the rinse liquid nozzle 43b dispenses the pure water toward the substrate W at 2000 mL/min while the spin chuck 41 rotates the substrate W at 1500 rpm. In the thirtieth step, the cup 42A is used. That is, as illustrated in FIG. 15A, in the thirtieth step, the pure water scattered from the peripheral edge of the substrate W is caught by the cup 42A.

In the thirty-first step, for 2 seconds, the rinse liquid nozzle 43b dispenses the pure water toward the substrate W at 2000 mL/min while the spin chuck 41 rotates the substrate W at 1500 rpm. In thirty-first step, the cup lifting mechanism 425 selects the cup to be used from the cup 42C to the cup 42A (see FIG. 15B). Thus, the pure water is caught by the cup 42C and flows into the upstream end of the collection pipe 51.

In the thirty-second step, for 0.2 seconds, while the spin chuck 41 rotates the substrate W at 1500 rpm, the rinse liquid nozzle 43b dispenses the pure water toward the substrate W at 2000 mL/min, and the IPA nozzle 43c dispenses the organic solvent toward the substrate W at 250 mL/min. As illustrated in FIG. 15C, also in the thirty-second step, the processing liquid scattered from the peripheral edge of the substrate W is caught by the cup 42C.

In thirty-third step, for 30 seconds, the IPA nozzle 43c dispenses the organic solvent toward the main surface of the substrate W at 250 mL/min while the spin chuck 41 rotates the substrate W at 1500 rpm. As illustrated in FIG. 15D, also in the thirty-third step, the processing liquid scattered from the peripheral edge of the substrate W is caught by the cup 42C. Through the thirty-second step and the thirty-third step, the pure water on the main surface of the substrate W is replaced with the organic solvent.

In the thirty-fourth step, the spin chuck 41 rotates the substrate W at 1500 rpm for 30 seconds. In the thirty-fourth step, the processing liquid is not supplied to the substrate W. As illustrated in FIG. 15E, also in the thirty-fourth step, the organic solvent scattered from the peripheral edge of the substrate W is caught by the cup 42C. In the thirty-fourth step, the substrate W is dried.

As described above, the cup 42C catches the processing liquid (the pure water and the organic solvent) scattered from the peripheral edge of the substrate W in thirty-first step to thirty-fourth step. The processing liquid flows into the upstream end of the collection pipe 51. Hereinafter, the period from the thirty-first step to the thirty-fourth step in Table 2 is referred to as a discharge period.

In Table 2, in the thirty-first step and the thirty-second step, the rinse liquid nozzle 43b dispenses the pure water toward the substrate W. For this reason, the pure water dispense amount is the sum of the amount (that is, the pure water film amount) of the pure water existing on the main surface of the substrate W at the start time point of the thirty-first step and the total amount (hereinafter, referred to as a pure water dispense amount) of the pure water discharged from the rinse liquid nozzle 43b during the discharge period. It can also be said that the start time point of the thirty-first step is a selecting start time point from the cup 42A to the cup 42C.

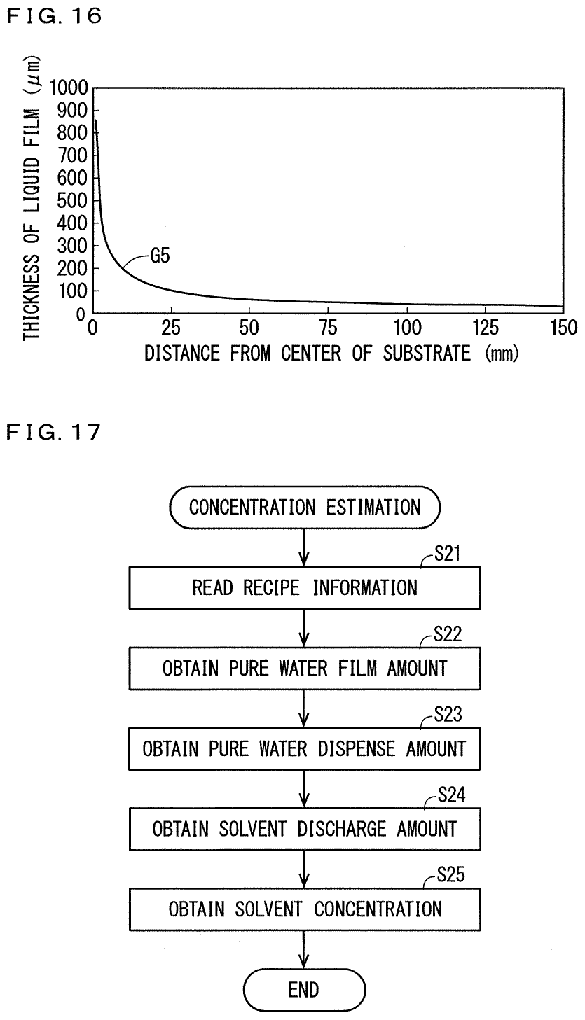

The pure water film amount depends on the rotation speed of the substrate W as described above. FIG. 16 is a graph illustrating an example of the distance from the center of the substrate W at each position on the substrate W and the thickness of the liquid film at each position. In FIG. 16, a graph G5 is illustrated. The rotation speed of the substrate W corresponding to the graph G5 is 1500 rpm. Information about the pure water film amount when the rotation speed of the substrate W is 1500 rpm is included in the correspondence relationship information D2.

The graph can also depend on the pure water flow rate of the thirtieth step before the cup selecting step. Accordingly, a graph may previously be obtained for each flow rate, and the pure water film amount may be obtained from the graph. In this case, the correspondence relationship information D2 includes a correspondence relationship between the combination of the rotation speed and the pure water flow rate and the pure water film amount.

The pure water dispense amount is the total amount of the pure water dispensed to the substrate W in the discharge period. For the pure water dispense amount, a time integral value of the pure water flow rate can be obtained. That is, the pure water dispense amount can be obtained by the sum of the product of the pure water flow rate of the pure water in each step and the required time (dispense time). In the example of Table 2, the pure water dispense amount is represented by 2000×(2+0.2)/60.

The solvent discharge amount can be considered to be equal to the dispense amount of the organic solvent to the substrate W in the discharge period. The discharge amount of the organic solvent in the discharge period can be obtained by the time integral value of the solvent flow rate. In the example of Table 2, the solvent discharge amount is represented by 250×(0.2+30)/60. The solvent discharge amount may be calculated by reducing the time integral value by a predetermined ratio.

FIG. 17 is a flowchart illustrating an example of the operation of the concentration estimator 601. First, the concentration estimator 601 reads the recipe information D1 from the storage 603 (step S21: reading step).

Subsequently, the concentration estimator 601 obtains the pure water film amount based on the recipe information D1 (step S22: pure water film amount calculation step). Specifically, the concentration estimator 601 specifies the step of starting the use of the cup 42C (for example, the thirty-first step in Table 1 or Table 2) from the recipe information D1, and specifies the rotation speed of the substrate W at the start time point of the step from the recipe information D1. The concentration estimator 601 may specify the rotation speed of the substrate W in the step as the rotation speed of the substrate W at the start time point of the step, or may specify the rotation speed of the substrate W in the step immediately before the step. Subsequently, the concentration estimator 601 reads the correspondence relationship information D2 from the storage 603. Then, the concentration estimator 601 obtains the pure water film amount (see FIG. 13C or FIG. 15B) based on the specified rotation speed and the correspondence relationship information D2.

When the correspondence relationship information D2 includes the correspondence relationship between the combination of the rotation speed and the pure water flow rate of the substrate W and the pure water film amount, the concentration estimator 601 may specify the pure water flow rate immediately before the step of starting the use of the cup 42C from the recipe information D1, and obtain the pure water film amount based on the specified rotation speed and pure water flow rate and the correspondence relationship information D2.

Furthermore, the concentration estimator 601 obtains the total amount (pure water dispense amount) of the pure water dispensed in the discharge period with respect to the recipe information D1 (step S23: pure water dispense amount calculation step). Specifically, the concentration estimator 601 specifies the step, in which the cup 42C is used and the pure water is dispensed, from the recipe information D1, and calculates the dispense amount of the pure water in the step by the product of the pure water flow rate and the required time. Then, the concentration estimator 601 calculates the sum of the dispense amounts in the respective steps as the pure water dispense amount.

In addition, the concentration estimator 601 obtains the total amount (solvent discharge amount) of the organic solvent dispensed in the discharge period with respect to the recipe information D1 (step S24: solvent discharge amount calculation step). Specifically, the concentration estimator 601 specifies the step, in which the cup 42C is used and the organic solvent is dispensed, from the recipe information D1, and calculates the dispense amount of the organic solvent in the steps by the product of the solvent flow rate and the required time. Then, the concentration estimator 601 calculates the sum of the dispense amounts in the respective steps as the solvent discharge amount. The concentration estimator 601 may calculate a value obtained by subtracting the sum by a predetermined ratio as the solvent discharge amount.

Subsequently, the concentration estimator 601 calculates the solvent concentration by dividing the solvent discharge amount by the sum of the pure water film amount, the pure water dispense amount, and the solvent discharge amount (step S25: solvent concentration calculation step).

As described above, the concentration estimator 601 calculates the solvent concentration based on the recipe information D1. For this reason, the concentration sensor that measures the solvent concentration is unnecessary, and the manufacturing cost of the substrate processing apparatus 100 can be reduced.

In the above example, the concentration estimator 601 obtains the pure water film amount based on the rotation speed of the substrate W, and calculates the solvent concentration based on the pure water film amount, the time integral value of the pure water flow rate of the pure water, and the time integral value of the solvent flow rate of the organic solvent. For this reason, the concentration estimator 601 can obtain the solvent concentration with higher accuracy. When the concentration estimator 601 obtains the pure water film amount based on the flow rate of the pure water and the rotation speed of the substrate W, the solvent concentration can be obtained with higher accuracy.

Sometimes the collection pipe connected to the cup 42C is branched into a plurality of pipes for each type of the processing liquid. For example, when the cup 42C is used for the organic solvent and another first processing liquid, the cup 42C is connected to the collection pipe 51 for the organic solvent and the collection pipe for the first processing liquid. The selecting valve part is also provided. The selecting valve part causes the pipe for the first processing liquid to communicate with the cup 42C when the first processing liquid is supplied to the substrate W, and causes the collection pipe 51 to communicate with the cup 42C when the organic solvent is supplied to the substrate W. In this case, a plurality of discharge ports are set in the cup 42C. In this case, the discharge port may be set in the recipe information D1. Then, the concentration estimator 601 may specify the step in which the discharge port (that is, the collection pipe 51) for the organic solvent is set, and obtain the pure water film amount, the pure water dispense amount, and the solvent discharge amount in the same manner as described above.

### <Measurement of Solvent Concentration Based on Concentration Sensor>

In the above example, the controller 6 calculates the solvent concentration of the mixed liquid discharged from the processing unit 4 based on the recipe information D1. However, the present embodiment is not necessarily limited thereto. The solvent concentration of the mixed liquid discharged from the processing unit 4 may be measured by the concentration sensor.

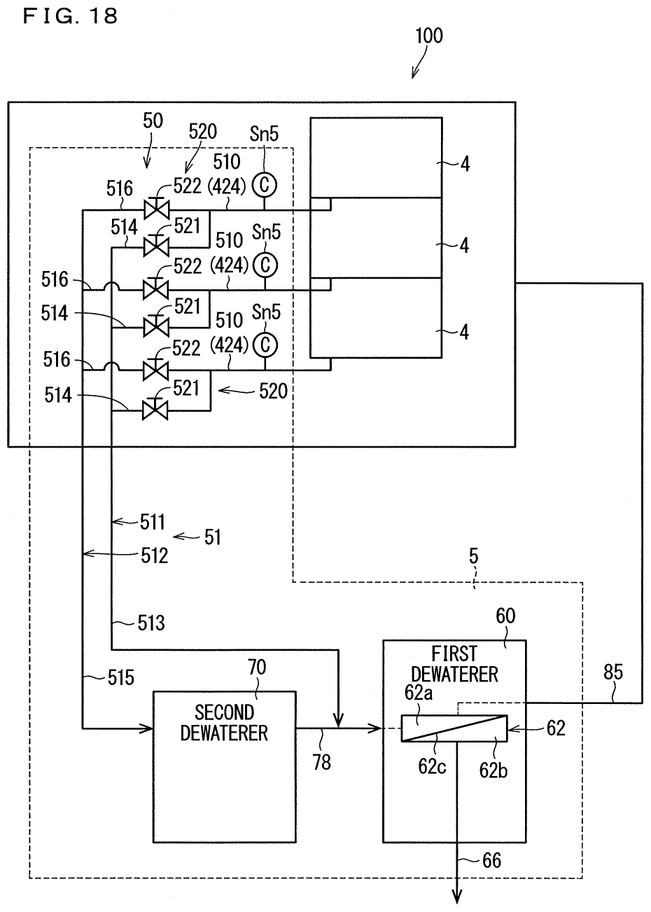

FIG. 18 is a view schematically illustrating a second example of the substrate processing apparatus 100 according to the fourth embodiment. In the second example, a concentration sensor Sn5 is provided in each common collection pipe 510. The concentration sensor Sn5 measures the solvent concentration of the mixed liquid flowing through the common collection pipe 510, and outputs a measurement result to the controller 6. The concentration sensor Sn5 may be a conductivity type concentration sensor, an optical type concentration sensor, or an ultrasonic type concentration sensor.

The controller 6 controls the collection destination selector 50 based on the solvent concentration measured by the concentration sensor Sn5. Specifically, the controller 6 compares the solvent concentration measured by the concentration sensor Sn5 with the selecting reference value, and causes the collection destination selector 50 to choose the second dewatering state when the solvent concentration is less than the selecting reference value, and the controller 6 causes the collection destination selector 50 to choose the first dewatering state when the solvent concentration is greater than or equal to the selecting reference value.

According to the second example, the concentration sensor Sn5 measures the solvent concentration, so that the controller 6 can acquire the solvent concentration of the mixed liquid with higher accuracy. For this reason, the controller 6 can more appropriately control the collection destination selector 50, and can more appropriately supply the mixed liquid to the first dewaterer 60 or the second dewaterer 70.

### Fifth Embodiment

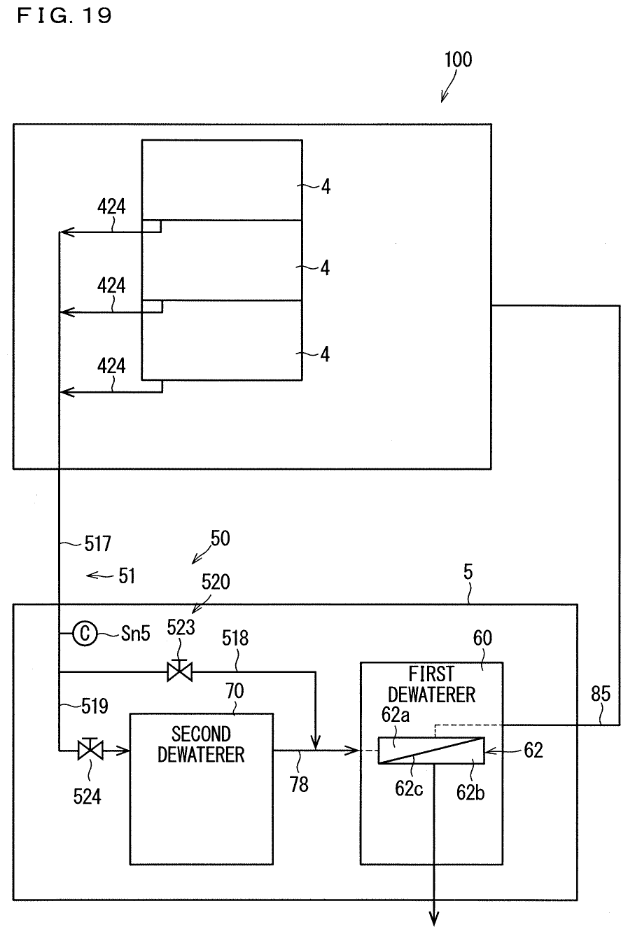

FIG. 19 is a view schematically illustrating an example of a substrate processing apparatus 100 according to a fifth embodiment. The substrate processing apparatus 100 according to the fifth embodiment is different from the substrate processing apparatus 100 according to the fourth embodiment in the configuration of the collection destination selector 50.

The collection destination selector 50 selects between the first dewatering state and the second dewatering state. In the fifth embodiment, the first dewatering state is a state in which the mixed liquid obtained by merging the mixed liquids from the plurality of processing units 4 is supplied to the first dewaterer 60, and the second dewatering state is a state in which the mixed liquid obtained by merging the mixed liquids from the plurality of processing units 4 is supplied to the second dewaterer 70.

The collection destination selector 50 includes the collection pipe 51 and a selecting valve part 520. The collection pipe 51 includes a common collection pipe 517, a first dewatering pipe 518, and a second dewatering pipe 519. The common collection pipe 517 is connected to each processing unit 4 (cup 42) through each cup-side collection pipe 424. In the common collection pipe 517, the mixed liquids from the plurality of processing units 4 can merge. The downstream end of the common collection pipe 517 is connected to the upstream end of the first dewatering pipe 518 and the upstream end of the second dewatering pipe 519. The downstream end of the first dewatering pipe 518 is connected to the first dewaterer 60, and the downstream end of the second dewatering pipe 519 is connected to the second dewaterer 70.

In the example of FIG. 19, the selecting valve part 520 includes a selecting valve 523 and a selecting valve 524. The selecting valve part 520 selects between the state in which the common collection pipe 517 communicates with the first dewaterer 60 (first dewatering state) and the state in which the common collection pipe 517 communicates with the second dewaterer 70 (second dewatering state). In the example of FIG. 19, the selecting valve 523 is interposed in the first dewatering pipe 518, and the selecting valve 524 is interposed in the second dewatering pipe 519.

When the controller 6 closes the selecting valve 523 and opens the selecting valve 524, the mixed liquid from the processing unit 4 flows through the common collection pipe 517 and the second dewatering pipe 519 in this order and is supplied to the second dewaterer 70. When the controller 6 opens the selecting valve 523 and closes the selecting valve 524, the mixed liquid from the processing unit 4 flows through the common collection pipe 517 and the first dewatering pipe 518 in this order and is supplied to the first dewaterer 60.

The controller 6 controls the collection destination selector 50 based on the solvent concentration of the mixed liquid flowing through the common collection pipe 517. In the example of FIG. 19, a concentration sensor Sn51 is provided in the common collection pipe 517. The concentration sensor Sn51 measures the solvent concentration of the mixed liquid flowing through the common collection pipe 517, and outputs the measurement result to the controller 6. An example of the configuration of the concentration sensor Sn51 is similar to that of the concentration sensor Sn5. The controller 6 causes the collection destination selector 50 to choose the second dewatering state when the solvent concentration measured by the concentration sensor Sn51 is a second value less than the concentration lower limit value of the first separation membrane 62c, and causes the collection destination selector 50 to choose the first dewatering state when the solvent concentration is the first value greater than or equal to the concentration lower limit value of the first separation membrane 62c. As a more specific example, the controller 6 may compare the solvent concentration measured by the concentration sensor Sn51 with the selecting reference value. The controller 6 causes the collection destination selector 50 to choose the second dewatering state when the solvent concentration is less than the selecting reference value, and causes the collection destination selector 50 to choose the first dewatering state when the solvent concentration is greater than or equal to the selecting reference value.

As described above, according to the fifth embodiment, the organic solvent collection part 5 selects the supply destination of the mixed liquid based on the solvent concentration of the mixed liquid obtained by merging the mixed liquids from the plurality of processing units 4. That is, in the fifth embodiment, the single collection destination selector 50 is provided corresponding to the plurality of processing units 4. Consequently, the manufacturing cost of the organic solvent collection part 5 can be reduced as compared with the fourth embodiment in which the plurality of collection destination selectors 50 is provided for the plurality of processing units 4 on a one-to-one basis.

In the above example, the concentration sensor Sn51 measures the solvent concentration, so that the controller 6 can acquire the solvent concentration of the mixed liquid with higher accuracy. For this reason, the controller 6 can more appropriately control the collection destination selector 50, and can more appropriately supply the mixed liquid to the first dewaterer 60 or the second dewaterer 70. Moreover, according to the fifth embodiment, the single concentration sensor Sn51 is provided corresponding to the plurality of processing units 4. For this reason, the manufacturing cost of the organic solvent collection part 5 can be reduced as compared with the fourth embodiment in which the plurality of concentration sensors Sn5 is provided for the plurality of processing units 4 on a one-to-one basis.

As described above, the organic solvent collection apparatus (organic solvent collection part 5), the substrate processing apparatus 100, and the organic solvent collection method have been described in detail, but the above description is an example in all aspects, and this disclosure is not limited thereto. In addition, the various modifications described above can be applied in combination as long as they do not contradict each other. Many modifications not illustrated can be envisaged without departing from the scope of the present disclosure.

For example, the organic solvent collection part 5 may include a filter that traps impurities in the recycling liquid. For example, the organic solvent collection part 5 may include a purification tank, a purification circulation pipe connected to the purification tank, and a selecting valve, a pump, and a filter interposed in the purification circulation pipe. Thus, the organic solvent collection part 5 can supply the recycling liquid having a low impurity concentration to the supply tank Tk3.

The present disclosure includes the following aspects.

A first aspect is an organic solvent collection apparatus including: a collection pipe through which a mixed liquid of an organic solvent and water discharged from a processing unit that processes a substrate flows; a first dewaterer including a first membrane separator including a first separation membrane having an application range of a solvent concentration, the first membrane separator separating water from the mixed liquid having the solvent concentration greater than or equal to a concentration lower limit value that is a lower limit value of the application range to increase the solvent concentration of the mixed liquid; and a second dewaterer that is provided at a preceding stage of the first dewaterer, the second dewaterer separating water from the mixed liquid discharged through the collection pipe to increase the solvent concentration of the mixed liquid to greater than or equal to the concentration lower limit value, and supplying the mixed liquid having the solvent concentration greater than or equal to the concentration lower limit value to the first dewaterer.

A second aspect is the organic solvent collection apparatus according to the first aspect, in which the second dewaterer includes a first liquid sending pipe through which the mixed liquid flows toward the first dewaterer, the first dewaterer includes a second liquid sending pipe through which the mixed liquid flows toward a supply tank for supply to the processing unit, and the first membrane separator is connected to a downstream end of the first liquid sending pipe and an upstream end of the second liquid sending pipe.

A third aspect is the organic solvent collection apparatus according to the first aspect, in which the first dewaterer includes: a concentration tank that stores the mixed liquid; a first circulation pipe that is connected to the concentration tank and provided with the first membrane separator; and a liquid sending part provided in the first circulation pipe.

A fourth aspect is the organic solvent collection apparatus according to any one of the first to third aspects, in which the second dewaterer includes at least one of a distillation column and an ultrasonic atomizer.

A fifth aspect is the organic solvent collection apparatus according to any one of the first to fourth aspects, in which the second dewaterer includes a second membrane separator having a second separation membrane, the concentration lower limit value of the application range of the solvent concentration of the second separation membrane is less than the concentration lower limit value of the first separation membrane, and a separation constant of the first separation membrane is greater than a separation constant of the second separation membrane.

A sixth aspect is the organic solvent collection apparatus according to the fifth aspect, in which the second dewaterer includes: a concentration tank that stores the mixed liquid flowing in from the collection pipe; a second circulation pipe that is connected to the concentration tank and provided with the second membrane separator; and a liquid sending part provided in the second circulation pipe.

A seventh aspect is the organic solvent collection apparatus according to the sixth aspect, further including a selecting valve part, in which the first dewaterer includes a first circulation pipe connected to the concentration tank, the first circulation pipe includes: a common circulation pipe provided with the liquid sending part; and a first individual pipe provided with the first membrane separator, the second circulation pipe includes: the common circulation pipe; and a second individual pipe provided with the second membrane separator, and the selecting valve part selects between a first circulation state in which the mixed liquid circulates through the concentration tank and the first circulation pipe and a second circulation state in which the mixed liquid circulates through the concentration tank and the second circulation pipe.

An eighth aspect is the organic solvent collection apparatus according to the seventh aspect, further including a controller that causes the selecting valve part to choose the second circulation state when the solvent concentration of the mixed liquid is a second value less than the concentration lower limit value of the first separation membrane, and causes the selecting valve part to choose the first circulation state when the solvent concentration of the mixed liquid is a first value greater than or equal to the concentration lower limit value.

A ninth aspect is the organic solvent collection apparatus according to any one of the first to sixth aspects, further including: a collection destination selector that selects between a second dewatering state in which the mixed liquid discharged from the processing unit is supplied to the second dewaterer and a first dewatering state in which the mixed liquid discharged from the processing unit is supplied to the first dewaterer while bypassing the second dewaterer; and a controller that causes the collection destination selector to choose the second dewatering state when the solvent concentration of the mixed liquid is a second value less than the concentration lower limit value of the first separation membrane, and causes the collection destination selector to choose the first dewatering state when the solvent concentration of the mixed liquid is a first value greater than or equal to the concentration lower limit value.

A tenth aspect is the organic solvent collection apparatus according to the ninth aspect, further including a storage that stores recipe information indicating a processing content of the substrate by the processing unit, in which the controller calculates the solvent concentration of the mixed liquid discharged from the processing unit based on the recipe information.

An eleventh aspect is the organic solvent collection apparatus according to the tenth aspect, in which the processing unit includes: a substrate holder that rotates the substrate while holding the substrate; a dispenser that sequentially dispenses pure water and an organic solvent to a main surface of the substrate held by the substrate holder; and a cup having a tubular shape surrounding the substrate holder and configured to catch liquid scattered from a peripheral edge of the substrate, an upstream end of the collection pipe is connected to the cup, a pure water flow rate and a dispense time of pure water dispensed to the substrate, a solvent flow rate and a dispense time of an organic solvent dispensed to the substrate, and a rotation speed of the substrate are set in the recipe information, the storage stores correspondence relationship information indicating a correspondence relationship between the rotation speed and a pure water film amount that is an amount of pure water on the main surface of the substrate, and the controller obtains the pure water film amount based on the rotation speed of the substrate specified based on the recipe information and the correspondence relationship information, and calculates the solvent concentration of the mixed liquid discharged from the processing unit based on the pure water film amount, a time integral value of the pure water flow amount, and a time integral value of the solvent flow rate.

A twelfth aspect is the organic solvent collection apparatus according to the ninth aspect, including a concentration sensor that measures the solvent concentration of the mixed liquid, in which the controller controls the collection destination selector based on the solvent concentration of the mixed liquid measured by the concentration sensor.

A thirteenth aspect is the organic solvent collection apparatus according to any one of the first to sixth aspects, including: a collection destination selector; and a controller that controls the collection destination selector, in which the collection pipe includes: a plurality of cup-side collection pipes connected to a plurality of the processing units; a common collection pipe connected to downstream ends of the plurality of cup-side collection pipes; a first dewatering pipe that connects a downstream end of the common collection pipe and the first dewaterer; and a second dewatering pipe that connects the downstream end of the common collection pipe and the second dewaterer, the collection destination selector selects between a first dewatering state in which the common collection pipe communicates with the first dewaterer through the first dewatering pipe and bypasses the second dewaterer to supply the mixed liquid to the first dewaterer and a second dewatering state in which the common collection pipe communicates with the second dewaterer through the second dewatering pipe to supply the mixed liquid to the second dewaterer, and the controller causes the collection destination selector to choose the first dewatering state when the solvent concentration of the mixed liquid flowing through the common collection pipe is a first value greater than or equal to the concentration lower limit value of the first separation membrane, and causes the collection destination selector to choose the second dewatering state when the solvent concentration of the mixed liquid flowing through the common collection pipe is a second value less than the concentration lower limit value.

A fourteenth aspect is a substrate processing apparatus includes the organic solvent collection apparatus according to any one of the first to thirteenth aspects and the processing unit.

A fifteenth aspect is an organic solvent collection method including: a first step of separating water from a mixed liquid of an organic solvent and water discharged from a processing unit that processes a substrate to increase a solvent concentration of the mixed liquid; and a second step of separating water from the mixed liquid using a first membrane separator including a first separation membrane having an application range of the solvent concentration after the first step to increase the solvent concentration of the mixed liquid, in which, in the first step, the solvent concentration of the mixed liquid is increased greater than or equal to a concentration lower limit value that is a lower limit value of the application range of the first separation membrane.

According to the first, fourteenth, and fifteenth aspects, even when the solvent concentration of the mixed liquid discharged from the processing unit is low, the second dewaterer increases the solvent concentration of the mixed liquid to greater than or equal to the concentration lower limit value of the first separation membrane. For this reason, the highly efficient first membrane separator can appropriately separate the water from the mixed liquid. Consequently, the organic solvent collection apparatus can increase the solvent concentration of the mixed liquid with high reliability and high efficiency.

According to the second aspect, the mixed liquid from the first liquid sending pipe passes through the first membrane separator and is supplied to the supply tank through the second liquid sending pipe. The first dewaterer does not circulate the mixed liquid, so that the time required for the operation by the first dewaterer can be shortened.

According to the third aspect, the size required for the first membrane separator can be reduced.

According to the fourth aspect, the concentration lower limit value of the second dewaterer is very low. For this reason, even when the solvent concentration of the mixed liquid discharged from the processing unit is very low, the second dewaterer can increase the solvent concentration of the mixed liquid to greater than or equal to the concentration lower limit value of the first separation membrane.

According to the fifth aspect, the second dewaterer can separate the water from the mixed liquid having the solvent concentration less than the concentration lower limit value of the first separation membrane to increase the solvent concentration of the mixed liquid. Then, after the second membrane separator increases the solvent concentration of the mixed liquid to greater than or equal to the concentration lower limit value of the first separation membrane, the first membrane separator including the first separation membrane having the high separation constant further increases the solvent concentration of the mixed liquid. For this reason, the organic solvent collection apparatus can increase the solvent concentration of the mixed liquid with higher efficiency.

According to the sixth aspect, the size required for the second membrane separator can be reduced.

According to the seventh aspect, the liquid sending part is shared by the first circulation pipe and the second circulation pipe, so that the manufacturing cost can be reduced.

According to the eighth aspect, when the solvent concentration of the mixed liquid in the concentration tank is low, the mixed liquid is circulated through the second circulation pipe. For this reason, the second membrane separator having the low concentration lower limit value separates the water from the mixed liquid to increase the solvent concentration. When the solvent concentration increases, the mixed liquid circulates through the first circulation pipe. For this reason, the first membrane separator having the high separation constant separates the water from the mixed liquid to increase the solvent concentration. Consequently, the organic solvent collection apparatus can increase the solvent concentration of the mixed liquid with high reliability and high efficiency.

According to the ninth and thirteenth aspects, the second dewaterer does not operate when the solvent concentration of the mixed liquid from the processing unit is high. For this reason, the power consumption by the second dewaterer can be avoided. On the other hand, when the solvent concentration of the mixed liquid from the processing unit is low, the second dewaterer operates. For this reason, the second dewaterer can supply the mixed liquid having the solvent concentration greater than or equal to the concentration lower limit value of the first separation membrane to the first dewaterer.

According to the tenth aspect, the provision of the concentration sensor is not required, so that the manufacturing cost can be reduced.

According to the eleventh aspect, the solvent concentration can be calculated with high accuracy.

According to the twelfth aspect, the solvent concentration can be obtained with high accuracy.

While the disclosure has been shown and described in detail, the foregoing description is in all aspects illustrative and not restrictive. It is therefore understood that numerous modifications and variations can be devised.

What is claimed is:

1. An organic solvent collection apparatus comprising:
a collection pipe through which a mixed liquid of an organic solvent and water discharged from a processing unit that processes a substrate flows;
a first dewaterer including a first membrane separator including a first separation membrane having an application range of a solvent concentration, the first membrane separator separating water from said mixed liquid having said solvent concentration greater than or equal to a concentration lower limit value that is a lower limit value of said application range to increase said solvent concentration of said mixed liquid; and
a second dewaterer that is provided at a preceding stage of said first dewaterer, the second dewaterer separating water from said mixed liquid discharged through said collection pipe to increase said solvent concentration of said mixed liquid to greater than or equal to said concentration lower limit value, and supplying said mixed liquid having said solvent concentration greater than or equal to said concentration lower limit value to said first dewaterer.

2. The organic solvent collection apparatus according to claim 1, wherein
said second dewaterer includes a first liquid sending pipe through which said mixed liquid flows toward said first dewaterer,
said first dewaterer includes a second liquid sending pipe through which said mixed liquid flows toward a supply tank for supply to said processing unit, and
said first membrane separator is connected to a downstream end of the first liquid sending pipe and an upstream end of the second liquid sending pipe.

3. The organic solvent collection apparatus according to claim 1, wherein
said first dewaterer includes:
a concentration tank that stores said mixed liquid;
a first circulation pipe connected to said concentration tank and provided with the first membrane separator; and
a liquid sending part provided in said first circulation pipe.

4. The organic solvent collection apparatus according to claim 1, wherein
said second dewaterer includes at least one of a distillation column and an ultrasonic atomizer.

5. The organic solvent collection apparatus according to claim 1, wherein
said second dewaterer includes a second membrane separator having a second separation membrane,
said concentration lower limit value of said application range of said solvent concentration of said second separation membrane is less than the concentration lower limit value of said first separation membrane, and
a separation constant of said first separation membrane is greater than a separation constant of said second separation membrane.

6. The organic solvent collection apparatus according to claim 5, wherein
said second dewaterer includes:
a concentration tank that stores said mixed liquid flowing in from the collection pipe;
a second circulation pipe that is connected to said concentration tank and provided with the second membrane separator; and
a liquid sending part provided in said second circulation pipe.

7. The organic solvent collection apparatus according to claim 6, further comprising
a selecting valve part, wherein
said first dewaterer includes a first circulation pipe connected to said concentration tank,
said first circulation pipe includes:
a common circulation pipe provided with said liquid sending part; and
a first individual pipe provided with said first membrane separator,
said second circulation pipe includes:
said common circulation pipe; and
a second individual pipe provided with said second membrane separator, and said selecting valve part selects between a first circulation state in which said mixed liquid circulates through said concentration tank and said first circulation pipe and a second circulation state in which said mixed liquid circulates through said concentration tank and said second circulation pipe.

8. The organic solvent collection apparatus according to claim 7, comprising
a controller that causes said selecting valve part to choose said second circulation state when said solvent concentration of said mixed liquid is a second value less than said concentration lower limit value of said first separation membrane, and causes said selecting valve part to choose said first circulation state when said solvent concentration of said mixed liquid is a first value greater than or equal to said concentration lower limit value.

9. The organic solvent collection apparatus according to claim 1, further comprising:
a collection destination selector that selects between a second dewatering state in which said mixed liquid discharged from said processing unit is supplied to said second dewaterer and a first dewatering state in which said mixed liquid discharged from said processing unit is supplied to said first dewaterer while bypassing said second dewaterer; and
a controller that causes said collection destination selector to choose said second dewatering state when said solvent concentration of said mixed liquid is a second value less than said concentration lower limit value of said first separation membrane, and causes said collection destination selector to choose said first dewatering state when said solvent concentration of said mixed liquid is a first value greater than or equal to said concentration lower limit value.

10. The organic solvent collection apparatus according to claim 9, comprising
a storage that stores recipe information indicating a processing content of said substrate by said processing unit, wherein
said controller calculates said solvent concentration of said mixed liquid discharged from said processing unit based on said recipe information.

11. The organic solvent collection apparatus according to claim 10, wherein said processing unit includes:
a substrate holder that rotates the substrate while holding said substrate;
a dispenser that sequentially dispenses pure water and an organic solvent to a main surface of said substrate held by said substrate holder; and
a cup having a tubular shape surrounding the substrate holder and configured to catch liquid scattered from a peripheral edge of the substrate,
an upstream end of said collection pipe is connected to said cup,
a pure water flow rate and a dispense time of pure water dispensed to said substrate, a solvent flow rate and a dispense time of an organic solvent dispensed to said substrate, and a rotation speed of said substrate are set in said recipe information,
said storage stores correspondence relationship information indicating a correspondence relationship between said rotation speed and a pure water film amount that is an amount of pure water on said main surface of said substrate, and
said controller obtains the pure water film amount based on said rotation speed of said substrate specified based on said recipe information and said correspondence relationship information, and calculates said solvent concentration of said mixed liquid discharged from said processing unit based on said pure water film amount, a time integral value of said pure water flow amount, and a time integral value of said solvent flow rate.

12. The organic solvent collection apparatus according to claim 9, comprising
a concentration sensor that measures said solvent concentration of said mixed liquid, wherein
said controller controls said collection destination selector based on said solvent concentration of said mixed liquid measured by said concentration sensor.

13. The organic solvent collection apparatus according to claim 1, comprising:
a collection destination selector; and
a controller that controls said collection destination selector, wherein
said collection pipe includes:
a plurality of cup-side collection pipes connected to a plurality of the processing units;
a common collection pipe connected to downstream ends of said plurality of cup-side collection pipes;
a first dewatering pipe that connects a downstream end of said common collection pipe and said first dewaterer; and
a second dewatering pipe that connects said downstream end of said common collection pipe and said second dewaterer,
said collection destination selector selects between a first dewatering state in which said common collection pipe communicates with said first dewaterer through said first dewatering pipe and bypasses said second dewaterer to supply said mixed liquid to said first dewaterer and a second dewatering state in which said common collection pipe communicates with said second dewaterer through said second dewatering pipe to supply said mixed liquid to said second dewaterer, and
said controller causes said collection destination selector to choose said first dewatering state when said solvent concentration of said mixed liquid flowing through said common collection pipe is a first value greater than or equal to said concentration lower limit value of said first separation membrane, and causes said collection destination selector to choose said second dewatering state when said solvent concentration of said mixed liquid flowing through said common collection pipe is a second value less than said concentration lower limit value.

14. A substrate processing apparatus comprising:
a processing unit that processes a substrate; and
an organic solvent collection apparatus including a collection pipe through which a mixed liquid of an organic solvent and water discharged from said processing unit flows, a first dewaterer including a first membrane separator including a first separation membrane having an application range of a solvent concentration, said first membrane separator separating water from said mixed liquid having said solvent concentration greater than or equal to a concentration lower limit value that is a lower limit value of said application range to increase said solvent concentration of said mixed liquid; and a second dewaterer that is provided at a preceding stage of said first dewaterer, said second dewaterer separating water from said mixed liquid discharged through said collection pipe to increase said solvent concentration of said mixed liquid to greater than or equal to said concentration lower limit value, and supplying said mixed liquid having said solvent concentration greater than or equal to said concentration lower limit value to said first dewaterer.

15. An organic solvent collection method comprising:
a first step of separating water from a mixed liquid of an organic solvent and water discharged from a processing unit that processes a substrate to increase a solvent concentration of said mixed liquid; and
a second step of separating water from said mixed liquid using a first membrane separator including a first separation membrane having an application range of said solvent concentration after said first step to increase said solvent concentration of said mixed liquid, wherein,
in the first step, said solvent concentration of said mixed liquid is increased greater than or equal to a concentration lower limit value that is a lower limit value of said application range of said first separation membrane.
